(19) United States
(12) Patent Application Publication
(10) Pub. No.: US 2024/0366830 A1
(43) Pub. Date: Nov. 7, 2024

(54) READY-TO-USE MONOMER-FREE ACRYLIC BONE CEMENT WITH ACCELERATED IN-SITE CURING PROPERTIES

(71) Applicant: SetBone Medical Ltd., Ness Ziona (IL)

(72) Inventors: Royi KAUFMANN, Hod-HaSharon (IL); Nimrod KAUFMANN, Modi'in-Maccabim-Re'ut (IL)

(73) Assignee: SetBone Medical Ltd., Ness Ziona (IL)

(21) Appl. No.: 18/687,349

(22) PCT Filed: Sep. 29, 2022
(86) PCT No.: PCT/IL2022/051032

§ 371 Date: Feb. 28, 2024

Related U.S. Application Data
(60) Provisional application No. 63/249,616, filed on Sep. 29, 2021.

(51) Int. Cl. A61L 24/04; A61B 17/88; A61L 24/00; A61L 24/02; B01F 23/50; B01F 23/57; B01F 35/221; B01F 35/71; B01F 101/20

(52) U.S. Cl. CPC A61L 24/043; A61B 17/8833; A61L 24/001; A61L 24/0015; A61L 24/0042; A61L 24/02; B01F 23/511; B01F 23/57; B01F 35/2211; B01F 35/7174; A61B 2017/8838; A61L 2300/406; A61L 2300/412; A61L 2300/414; A61L 2300/44; A61L 2430/02; B01F 2101/20; B01F 2215/044

(57) ABSTRACT

The present invention relates to a medical grade bone cement having at least two mixable components, the bone cement comprising: a first component comprising at least 20% by weight of a first polymer material in which at least 40% of the functional groups are methacrylates; and a second component comprising at least one initiator selected to cause cross-linking of said first polymer material, wherein a balance of said second component comprises at least 40% by weight of a second polymer material that does not react with said initiator; said second component being a paste.

### RELATED APPLICATION

This application claims the benefit of priority of U.S. Provisional Patent Application No. 63/249,616 filed on 29 Sep. 2021, the contents of which are incorporated herein by reference in their entirety.

### FIELD AND BACKGROUND OF THE INVENTION

The present invention, in some embodiments thereof, relates to medical grade and non-medical grade materials, which are configured to change their property following stimulation.

Percutaneous vertebral augmentation is a minimally invasive procedure to augment a vertebra that has fractured because of osteoporosis or injury to the spine. In order to stabilize the fractured vertebra and alleviate pain, a needle is inserted through a drill in the spinal pedicle into the vertebral body. In a vertebroplasty treatment a liquid form of methyl-methacrylate/polymethylmethacrylate (PMMA) bone cement is injected through the needle directly into the fractured vertebra, where it hardens and stabilizes the fractured bone. In a kyphoplasty treatment, prior to application of the bone cement, an uninflated balloon is inserted into the cancellous bone of the vertebral body, inflated to restore the vertebra height and then removed leaving behind a void to be filled with the cement. The most adverse event of the treatment is cement leakage outside the vertebral body. If the cement leaks into the spinal canal, it may compress the spinal cord and nerves, resulting in new pain and neurologic problems. From the physician's viewpoint, the main drawback of the PMMA cement is that it starts to harden immediately upon preparation. Therefore, the cement viscosity varies during the work and the total working time is limited to a few minutes, which can potentially lead to improper filling of the fractured vertebrae.

Additional background art includes International Patent Application Publication No. WO2021/038562 disclosing a deformable body formed of at least 20% by volume of a polymer material in which individual polymer backbones have a plurality of functional groups capable of cross-linking to form a cross-link; said polymer material provided in a first less-cross-linked configuration; wherein said polymer material, upon application of a suitable stimuli, said stimuli causes cross-linking of said functional groups to form cross-linking between said polymer backbones such that said polymer material is in a second more-cross-linked configuration.

U.S. Pat. No. 5,264,215A discloses “a bone cement composition comprising polyalkyl methacrylate derived from methacrylate having an alkyl group of 1-4 carbon atoms, hydroxyapatite, alkyl methacrylate having an alkyl group of 1-4 carbon atoms, 4-(2-methacryloyloxyethyl)trimellitic acid or anhydride thereof, and a polymerization initiator. Also disclosed are a cured product of the composition, an implant material using the composition and a process for the preparation of the implant material”.

U.S. Pat. No. 5,538,514A discloses “a method for molding bone cement to a prosthetic implant in which a cement mixture absent any amine initiators is molded about the implant and polymerization is initiated by exposing the cement mixture to a radiation source. The method eliminates the manufacturing timing problems of molding a polymerizing bone cement mixture to an implant”.

U.S. Pat. No. 8,475,536B2 discloses “Biomedical implants (e.g., orthopedic implants) with modified surfaces that can enhance a cement bond's strength (e.g., tensile, shear, and/or fatigue) are disclosed, along with methods of manufacturing and using such implants. The implants can exhibit a variety of physical, chemical, or process-derived features which can enhance cement bonding. For instance, the implant surface can exhibit particular roughness values, and/or be substantially free of non-native material. Processes for producing such implants can include providing a first roughened implant surface, which can be produced, for example, by particle blasting. A treatment formulation can be applied to the first roughened surface to create a second roughened surface that exhibits enhanced cement bonding properties relative to the first roughened surface. In some instances, the first roughened surface and the second roughened surface can exhibit substantially similar Ra values. The second roughened surface can exhibit a negative Rsk value.”

U.S. Pat. No. 8,926,710B2 discloses “osteoconductive bone graft materials. These compositions contain injectable cements and demineralized bone matrix fibers. The combination of these materials enables the filling of a bone void while balancing strength and resorption”.

U.S. Pat. No. 8,834,845B2 discloses “a bioactive PMMA (polymethylmethacrylate) bone cement contains a powder component and a reactive monomer liquid, wherein the powder component and the reactive monomer liquid when mixed with one another react with one another and form a polymer-based solid material. The powder component contains particulate polymer powder of polymethylmethacrylates; a radical starter; and anionic copolymer nanoparticles. The anionic copolymer nanoparticles are distributed in nano-particulate form within the particulate powder component or coated as a film on particles of the particulate polymer powder”.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A medical grade bone cement having at least two mixable components, the bone cement comprising:
a. a first component comprising at least 20% by weight of a first polymer material in which at least 40% of monomeric units comprise a methacrylate group; and
b. a second component comprises at least one initiator selected to cause cross-linking of said first polymer material, wherein a balance of said second component comprises at least 50% by weight of a second polymer material that does not react with said initiator; said second component being a paste.

Example 2. The medical grade bone cement according to example 1, wherein said first component and said second component are characterized by having similar viscosities within a factor of 10.

Example 3. The medical grade bone cement according to example 1 or example 2, wherein said first component is less than 10% by weight of monomers.

Example 4. The medical grade bone cement according to any one of examples 1-3, wherein said first component is monomer-free.

Example 5. The medical grade bone cement according to any one of examples 1-4, wherein said first component comprises at least from about 20% to about 80% by weight of said first polymer material.

Example 6. The medical grade bone cement according to any one of examples 1-5, wherein said second component comprises at least from about 40% to about 100% by weight of said second polymer material.

Example 7. The medical grade bone cement according to any one of examples 1-6, wherein said bone cement is provided as a ready to use bone cement in a dedicated implementation device.

Example 8. The medical grade bone cement according to any one of examples 1-7, wherein said first polymer material comprises a Polyvinyl alcohol (PVA) based polymer modified with methacrylic groups.

Example 9. The medical grade bone cement according to example 8, wherein said first polymer material comprising said PVA based polymer modified with methacrylic groups is PVGMA.

Example 10. The medical grade bone cement according to example 8, wherein a methacrylate modification along said PVA is characterized by a ratio of from about 1:100 to about 20:1.

Example 11. The medical grade bone cement according to example 8, wherein a methacrylate modification along said PVA is characterized by addition of branches of poly glycidyl methacrylate ether (PGMAE).

Example 12. The medical grade bone cement according to any one of examples 1-11, wherein a minimum quantity of PGMAE in said first polymer material has a 1:10 ratio of PGMAE/PVA and said PGMAE comprises a length of 4 units.

Example 13. The medical grade bone cement according to any one of examples 1-12, wherein said first polymer material is 100% PGMAE.

Example 14. The medical grade bone cement according to any one of examples 1-13, wherein said first component comprises a solvent selected from the group consisting of poly ethylene glycol di/mono-methacrylate, poly propylene glycol di/mono-methacrylate, poloxamer di/mono-methacrylate and any combination thereof.

Example 15. The medical grade bone cement according to any one of examples 1-14, wherein said first component comprises a PEG/PPG based solvent.

Example 16. The medical grade bone cement according to any one of examples 1-15, wherein said first component comprises a linker selected from the group consisting of Bisphenol A Glycidyl Dimethacrylate (bisGMA), bisphenol A Ethylene Glycol Dimethacrylate (bisEMA), triethylene glycol dimethacrylate (TEGDMA), Ethylene glycol dimethacrylate (EGDMA), Butandiol diacrylate (BDDA), Urethane Dimethacrylate (UDMA) and any combination thereof.

Example 17. The medical grade bone cement according to any one of examples 1-16, wherein said first component comprises at least one additive selected from the group consisting of a radiopaque material, an inhibitor, water and any combination thereof.

Example 18. The medical grade bone cement according to any one of examples 1-17, wherein said first component is characterized by the ability to absorb water.

Example 19. The medical grade bone cement according to example 18, wherein ether groups between methacrylic functional groups provide said ability to absorb water.

Example 20. The medical grade bone cement according to any one of examples 1-19, wherein said first component is characterized by an inability to absorb water.

Example 21. The medical grade bone cement according to example 18, wherein said PVA provide said ability to absorb water.

Example 22. The medical grade bone cement according to any one of examples 1-21, wherein said second polymer material is selected from the group consisting of Pluronic P123, Pluronic P105, Pluronic P85, paste PEG1000, paste PPG8000, mixture of short liquid and long solid PEGs PPGs, poloxamers and any combination thereof.

Example 23. The medical grade bone cement according to any one of examples 1-22, wherein said second polymer material is a bio-degradable polymer.

Example 24. The medical grade bone cement according to example 23, wherein said bio-degradable polymer are one or more of polyesters and polysaccharides.

Example 25. The medical grade bone cement according to example 23, wherein said bio-degradable polymer is a cross-linkable polymer configured to be part of the cross-linking polymerization process.

Example 26. The medical grade bone cement according to example 25, wherein said cross-linkable polymer is poly propylene fumarate (PPF).

Example 27. The medical grade bone cement according to any one of examples 1-26, wherein said at least one initiator is selected from the group consisting of sodium persulfate (NPS), ammonium persulfate (APS), potassium persulfate (KPS) and any combination thereof.

Example 28. The medical grade bone cement according to any one of examples 1-27, wherein said second component comprises at least one co-initiator.

Example 29. The medical grade bone cement according to example 28, wherein said co-initiator is one or more of ascorbic acid and a modified molecule based on ascorbic acid.

Example 30. The medical grade bone cement according to any one of examples 1-29, wherein a ratio of co-initiation to initiation is from about 0.1:1 to about 1:1.

Example 31. The medical grade bone cement according to any one of examples 1-30, wherein said second component comprises at least one stabilizer.

Example 32. The medical grade bone cement according to any one of examples 1-31, wherein said second component comprises at least one additive.

Example 33. The medical grade bone cement according to example 32, wherein said at least one additive is one or more of a pharmaceutical, antibiotics, stem cells and bone growth factors.

Example 34. The medical grade bone cement according to any one of examples 1-33, wherein a concentration of said initiator is from about 1:10 to about 1:500 in relation to methacrylic functional groups.

Example 35. The medical grade bone cement according to any one of examples 1-34, wherein a ratio between said first component and said second component is from about 1:10 to about 1:1.

Example 36. The medical grade bone cement according to any one of examples 1-35, wherein said medical grade bone cement, after being mixed, is characterized by having a low viscosity level of from about 10 Pa·s to about 100 Pa·s.

Example 37. The medical grade bone cement according to any one of examples 1-36, wherein said medical grade bone cement, after being mixed, is characterized by having a medium viscosity level of from about 100 Pa·s to about 500 Pa·s.

Example 38. The medical grade bone cement according to any one of examples 1-37, wherein said medical grade bone cement, after being mixed, is characterized by having a high viscosity level of from about 500 Pa·s to about 1500 Pa·s.

Example 39. The medical grade bone cement according to any one of examples 1-38, wherein said medical grade bone cement is characterized by a working time from about 1 min to about 40 min at room temperature.

Example 40. The medical grade bone cement according to example 39, wherein said medical grade bone cement is characterized by a hardening time starting from about an end of said working time to about 24 hours.

Example 41. The medical grade bone cement according to example 39, wherein said medical grade bone cement is characterized by achieving a non-deformable stage after from about 1 minute to about 5 minutes from about an end of said working time.

Example 42. The medical grade bone cement according to any one of examples 1-41, wherein a polymerization process is affected by temperature.

Example 43. The medical grade bone cement according to any one of examples 1-42, wherein a polymerization process is affected by moisture.

Example 44. The medical grade bone cement according to any one of examples 1-43, wherein exposure of said bone cement to temperatures of from about 35° C. to about 40° C. accelerates the polymerization process time by a factor of about 2 to about 5 compared to process time at temperatures of from about 20° C. to about 25° C.

Example 45. The medical grade bone cement according to any one of examples 1-44, wherein exposure of said bone cement to moisture accelerates the polymerization process time by a factor of about 2 to about 5.

Example 46. The medical grade bone cement according to any one of examples 1-45, wherein concomitant exposure of said bone cement to moisture and to temperatures of from about 35° C. to about 40° C. accelerates the polymerization process time by a factor of about 2 to about 25. Example 47. The medical grade bone cement according to any one of examples 1-46, wherein said medical grade bone cement comprises a color indicator for a polymerization process.

Example 48. A method of polymerizing a bone cement, comprising providing said bone cement with two distinct polymerization reactions, wherein a first polymerization reaction is characterized by being a fast polymerization reaction that is not temperature dependent, while a second polymerization reaction is characterized by being a slow polymerization reaction that is temperature dependent.

Example 49. A delivery device for a bone cement comprising:
a. a mixing unit comprising:
i. at least one cartridge comprising at least two chambers; each chamber from said at least two chambers containing a part of said bone cement; said at least one cartridge comprising a pushing mechanism of said parts of said bone cement; wherein said pushing mechanism is configured to move a same distance within said at least two chambers; and
ii. a mixer connected to said at least two chambers and configured to receive parts of said bone cement contained in said at least two chambers;
b. a control unit comprising at least one actuator for actuating at least one actuating mechanism to cause said parts of said bone cement contained in said at least two chambers to exit from said at least two chambers into said mixer;
c. a connector, connected on one side to said mixing unit and on a second side to said control unit; said connector configured to transmit actuation actions from said control unit to said mixing unit.

Example 50. The delivery device according to example 49, wherein said at least one actuating mechanism comprises one or more of hydraulic, mechanical and electrical actuation mechanisms.

Example 51. The delivery device according to example 49, wherein said pushing mechanism is a plunger configured to push said parts of said bone cement out of said at least two chambers.

The following is another non-exclusive list including some examples of embodiments of the invention. The invention also includes embodiments which include fewer than all the features in an example and embodiments using features from multiple examples, also if not expressly listed below.

Example 1. A medical grade bone cement having at least two mixable components, the bone cement comprising:
a. a first component comprising at least 20% by weight of a first polymer material in which at least 40% of the monomeric units comprise a methacrylate group; and
b. a second component comprises at least one initiator selected to cause cross-linking of said first polymer material, wherein a balance of said second component comprises at least 50% by weight of a second polymer material that does not react with said initiator; said second component being a paste.

Example 2. The medical grade bone cement according to example 1, wherein said first component and said second component are characterized by having similar viscosities within a factor of 10.

Example 3. The medical grade bone cement according to example 1 or example 2, wherein said first component is less than 10% by weight of monomers.

Example 4. The medical grade bone cement according to any one of examples 1-3, wherein said first component is monomer-free.

Example 5. The medical grade bone cement according to any one of examples 1-4, wherein said first component comprises at least from about 20% to about 80% by weight of said first polymer.

Example 6. The medical grade bone cement according to any one of examples 1-5, wherein said second component comprises at least from about 40% to about 100% by weight of said second polymer.

Example 7. The medical grade bone cement according to any one of examples 1-6, wherein said second component does not require pre-processing actions before being used.

Example 8. The medical grade bone cement according to any one of examples 1-7, wherein said bone cement is provided as a ready to use bone cement in a dedicated implementation device.

Example 9. The medical grade bone cement according to any one of examples 1-8, wherein said first polymer material comprises a Polyvinyl alcohol (PVA) based polymer modified with methacrylic groups.

Example 10. The medical grade bone cement according to any one of examples 1-9, wherein said first polymer material comprising said PVA based polymer modified with methacrylic groups is PVGMA.

Example 11. The medical grade bone cement according to any one of examples 1-10, wherein said PVA is characterized by a degree of hydrolyzation of from about 40% to about 99.9%.

Example 12. The medical grade bone cement according to any one of examples 1-11, wherein a methacrylate modification along said PVA is characterized by a ratio of from about 1:100 to about 20:1.

Example 13. The medical grade bone cement according to any one of examples 1-12, wherein a methacrylate modification along said PVA is characterized by addition of branches of poly glycidyl methacrylate ether (PGMAE).

Example 14. The medical grade bone cement according to any one of examples 1-13, wherein a minimum quantity of PGMAE in said first polymer material is about 3 times more by weight than said PVA.

Example 15. The medical grade bone cement according to any one of examples 1-14, wherein a minimum quantity of PGMAE in said first polymer material is about 1.2 times larger by molar ratio than said PVA.

Example 16. The medical grade bone cement according to any one of examples 1-15, wherein a minimum quantity of PGMAE in said first polymer material has a 1:10 ratio of PGMAE/PVA and said PGMAE comprises a length of 4 units.

Example 17. The medical grade bone cement according to any one of examples 1-16, wherein said first polymer material is 100% PGMAE.

Example 18. The medical grade bone cement according to any one of examples 1-17, wherein said first component comprises a solvent selected from the group consisting of poly ethylene glycol di/mono-methacrylate, poly propylene glycol di/mono-methacrylate, poloxamer di/mono-methacrylate and any combination thereof.

Example 19. The medical grade bone cement according to any one of examples 1-18, wherein said first component comprises a PEG/PPG based solvent.

Example 20. The medical grade bone cement according to any one of examples 1-19, wherein said first component comprises a linker selected from the group consisting of Bisphenol A Glycidyl Dimethacrylate (bisGMA), bisphenol A Ethylene Glycol Dimethacrylate (bisEMA), triethylene glycol dimethacrylate (TEGDMA), Urethane Dimethacrylate (UDMA) and any combination thereof.

Example 21. The medical grade bone cement according to any one of examples 1-20, wherein said first component comprises at least one additive selected from the group consisting of a radiopaque material, an inhibitor, water and any combination thereof.

Example 22. The medical grade bone cement according to any one of examples 1-21, wherein said first component is characterized by the ability to absorb water.

Example 23. The medical grade bone cement according to any one of examples 1-22, wherein ether groups between said methacrylic functional groups provide said ability to absorb water.

Example 24. The medical grade bone cement according to any one of examples 1-23, wherein said PVA provide said ability to absorb water.

Example 25. The medical grade bone cement according to any one of examples 1-24, wherein said second polymer is selected from the group consisting of Pluronic P123, Pluronic P105, Pluronic P85, paste PEG1000, paste PPG8000, mixture of short liquid and long solid PEGs PPGs, poloxamers and any combination thereof.

Example 26. The medical grade bone cement according to any one of examples 1-25, wherein said second polymer is a bio-degradable polymer.

Example 27. The medical grade bone cement according to any one of examples 1-26, wherein said bio-degradable polymer is one or more of polyesters and polysaccharides.

Example 28. The medical grade bone cement according to any one of examples 1-27, wherein said at least one initiator is selected from the group consisting of sodium persulfate (NPS), ammonium persulfate (APS), potassium persulfate (KPS) and any combination thereof.

Example 29. The medical grade bone cement according to any one of examples 1-28, wherein said second component comprises at least one co-initiator.

Example 30. The medical grade bone cement according to any one of examples 1-29, wherein said co-initiator is ascorbic acid.

Example 31. The medical grade bone cement according to any one of examples 1-30, wherein said co-initiator is a modified molecule based on ascorbic acid.

Example 32. The medical grade bone cement according to any one of examples 1-31, wherein a ratio of co-initiation to initiation is from about 0.1:1 to about 1:1.

Example 33. The medical grade bone cement according to any one of examples 1-32, wherein said second component comprises at least one stabilizer.

Example 34. The medical grade bone cement according to any one of examples 1-33, wherein said second component comprises at least one additive.

Example 35. The medical grade bone cement according to any one of examples 1-34, wherein said at least one additive is a pharmaceutical.

Example 36. The medical grade bone cement according to any one of examples 1-35, wherein said at least one additive is selected from the group consisting of antibiotics, stem cells, bone growth factors and any combination thereof.

Example 37. The medical grade bone cement according to any one of examples 1-36, wherein a concentration of said initiator is from about 1:10 to about 1:500 in relation to said methacrylic functional groups.

Example 38. The medical grade bone cement according to any one of examples 1-37, wherein a ratio between said first component and said second component is from about 1:10 to about 1:1.

Example 39. The medical grade bone cement according to any one of examples 1-38, wherein said medical grade bone cement, after being mixed, is characterized by having a low viscosity level of from about 10 Pa·s to about 100 Pa·s.

Example 40. The medical grade bone cement according to any one of examples 1-39, wherein said medical grade bone cement, after being mixed, is characterized by having a medium viscosity level of from about 100 Pa·s to about 500 Pa·s.

Example 41. The medical grade bone cement according to any one of examples 1-40, wherein said medical grade bone cement, after being mixed, is characterized by having a high viscosity level of from about 500 Pa·s to about 1500 Pa·s.

Example 42. The medical grade bone cement according to any one of examples 1-41, wherein said medical grade bone cement is characterized by a working time from about 1 min to about 40 min at room temperature.

Example 43. The medical grade bone cement according to any one of examples 1-42, wherein said medical grade bone cement is characterized by a hardening time starting from about an end of said working time to about 24 hours.

Example 44. The medical grade bone cement according to any one of examples 1-43, wherein said medical grade bone cement is characterized by achieving a non-deformable stage after from about 1 minute to about 5 minutes from about an end of said working time.

Example 45. The medical grade bone cement according to any one of examples 1-44, wherein a polymerization process is affected by temperature.

Example 46. The medical grade bone cement according to any one of examples 1-45, wherein a polymerization process is affected by moisture.

Example 47. The medical grade bone cement according to any one of examples 1-46, wherein exposure of said bone cement to temperatures of from about 35° C. to about 40° C. accelerates the polymerization process time by a factor of about 2 to about 5 compared to process time at temperatures of from about 20° C. to about 25° C.

Example 48. The medical grade bone cement according to any one of examples 1-47, wherein exposure of said bone cement to moisture accelerates the polymerization process time by a factor of about 2 to about 5.

Example 49. The medical grade bone cement according to any one of examples 1-48, wherein concomitant exposure of said bone cement to moisture and to temperatures of from about 35° C. to about 40° C. accelerates the polymerization process time by a factor of about 2 to about 25.

Example 50. The medical grade bone cement according to any one of examples 1-49, wherein said medical grade bone cement comprises a color indicator for the polymerization process.

Example 51. The medical grade bone cement according to any one of examples 1-50, wherein said working time is affected by temperature.

Example 52. The medical grade bone cement according to any one of examples 1-51, wherein said working time process is affected by moisture.

Example 53. The medical grade bone cement according to any one of examples 1-52, wherein exposure of said bone cement to temperatures of from about 35° C. to about 40° C. reduces said working time by a factor of about 2 to about 5 compared to said working time at room temperatures of from about 20° C. to about 25° C.

Example 54. The medical grade bone cement according to any one of examples 1-53, wherein exposure of said bone cement to moisture reduces said working time by a factor of about 2 to about 5.

Example 55. The medical grade bone cement according to any one of examples 1-54, wherein concomitant exposure of said bone cement to moisture and to temperatures of from about 35° C. to about 40° C. reduces the working time by a factor of about 2 to about 25.

Example 56. The medical grade bone cement according to any one of examples 1-55, wherein said medical grade bone cement comprises a color indicator for said working time.

Example 57. The medical grade bone cement according to any one of examples 1-56, wherein said first polymer material is hydrophilic at least in part.

Example 58. The medical grade bone cement according to any one of examples 1-57, further comprising a second component comprising a hydrophilic initiator.

Example 59. The medical grade bone cement according to any one of examples 1-58, wherein said first polymer material is hydrophilic only in part.

Example 60. The medical grade bone cement according to any one of examples 1-59, further comprising a second component comprising a hydrophilic initiator.

Example 61. The medical grade bone cement according to any one of examples 1-60, wherein said first polymer material is hygroscopic at least in part.

Example 62. The medical grade bone cement according to any one of examples 1-61, wherein said first polymer material is hygroscopic only in part.

Example 63. The medical grade bone cement according to any one of examples 1-62, wherein said medical grade bone cement is stored at room temperature before being used.

Example 64. The medical grade bone cement according to any one of examples 1-63, wherein said medical grade bone cement comprises a shelf-life of from about 1 year to about 10 years.

Example 65. The medical grade bone cement according to any one of examples 1-64, wherein said components and ingredients thereof are divided into more than two parts before mixing.

Example 66. The medical grade bone cement according to any one of examples 1-65, wherein said bone cement changes color during the polymerization process thereby providing an intrinsic curing indicator.

Example 67. The medical grade bone cement according to any one of examples 1-66, wherein said polymerization process is characterized by being a low exothermal polymerization process.

Example 68. A bone cement system comprising:
a. an applicator comprising two cartridges;
b. a first component in one first cartridge and a second component in one second cartridge;
wherein said first component comprises at least 20% by weight of a first polymer material in which at least 40% of the monomeric units comprise a methacrylate group; and
wherein said second component comprises at least one initiator selected to cause cross-linking of said first polymer material, wherein a balance of said second component comprises at least 50% by weight of a second polymer material that does not react with said initiator; said second component being a paste; and
c. a mixer comprising a channel in fluid communication with said two cartridges; said channel comprising a plurality of mixing levels for mixing said first component and said second component.

Example 69. The bone cement system according to example 68, wherein said first component and said second component are characterized by having similar viscosities within a factor of 10.

Example 70. The bone cement system according to example 68 or example 69, wherein said first component is less than 10% by weight of monomers.

Example 71. The bone cement system according to any one of examples 68-70, wherein said first component is monomer-free.

Example 72. The bone cement system according to any one of examples 68-71, wherein said first component comprises at least from about 20% to about 80% by weight of said first polymer.

Example 73. The bone cement system according to any one of examples 68-72, wherein said second component comprises at least from about 40% to about 100% by weight of said second polymer.

Example 74. The bone cement system according to any one of examples 68-73, wherein said second component does not require pre-processing actions before being used.

Example 75. The bone cement system according to any one of examples 68-74, wherein said bone cement is provided as a ready to use bone cement in a dedicated implementation device.

Example 76. The bone cement system according to any one of examples 68-75, wherein said first polymer material comprises a Polyvinyl alcohol (PVA) based polymer modified with methacrylic groups.

Example 77. The bone cement system according to any one of examples 68-76, wherein said first polymer material comprising said PVA based polymer modified with methacrylic groups is PVGMA.

Example 78. The bone cement system according to any one of examples 68-77, wherein said PVA is characterized by a degree of hydrolyzation of from about 40% to about 99.9%.

Example 79. The bone cement system according to any one of examples 68-78, wherein a methacrylate modification along said PVA is characterized by a ratio of from about 1:100 to about 20:1.

Example 80. The bone cement system according to any one of examples 68-79, wherein a methacrylate modification along said PVA is characterized by addition of branches of poly glycidyl methacrylate ether (PGMAE).

Example 81. The bone cement system according to any one of examples 68-80, wherein a minimum quantity of PGMAE in said first polymer material is about 3 times more by weight than said PVA.

Example 82. The bone cement system according to any one of examples 68-81, wherein a minimum quantity of PGMAE in said first polymer material is about 1.2 times larger by molar ratio than said PVA.

Example 83. The bone cement system according to any one of examples 68-82, wherein a minimum quantity of PGMAE in said first polymer material is in a ratio of 1:10 of PGMAE/PVA and said PGMAE comprises a length of 4 units.

Example 84. The bone cement system according to any one of examples 68-83, wherein said first polymer material is 100% PGMAE.

Example 85. The bone cement system according to any one of examples 68-84, wherein said first component comprises a solvent selected from the group consisting of poly ethylene glycol di/mono-methacrylate, poly propylene glycol di/mono-methacrylate, poloxamer di/mono-methacrylate and any combination thereof.

Example 86. The bone cement system according to any one of examples 68-85, wherein said first component comprises a PEG/PPG based solvent.

Example 87. The bone cement system according to any one of examples 68-86, wherein said first component comprises a linker selected from the group consisting of Bisphenol A Glycidyl Dimethacrylate (bisGMA), bisphenol A Ethylene Glycol Dimethacrylate (bisEMA), triethylene glycol dimethacrylate (TEGDMA), Urethane Dimethacrylate (UDMA) and any combination thereof.

Example 88. The bone cement system according to any one of examples 68-87, wherein said first component comprises at least one additive selected from the group consisting of a radiopaque material, an inhibitor, water and any combination thereof.

Example 89. The bone cement system according to any one of examples 68-88, wherein said first component is characterized by the ability to absorb water.

Example 90. The bone cement system according to any one of examples 68-89, wherein ether groups between said methacrylic functional groups provide said ability to absorb water.

Example 91. The bone cement system according to any one of examples 68-90, wherein said PVA provides said ability to absorb water.

Example 92. The bone cement system according to any one of examples 68-91, wherein said second polymer is selected from the group consisting of Pluronic P123, Pluronic P105,
Pluronic P85, paste PEG1000, paste PPG8000, mixture of short liquid and long solid PEGs PPGs, poloxamers and any combination thereof.

Example 93. The bone cement system according to any one of examples 68-92, wherein said second polymer is a bio-degradable polymer.

Example 94. The bone cement system according to any one of examples 68-93, wherein said bio-degradable polymer are one or more of polyesters and polysaccharides.

Example 95. The bone cement system according to any one of examples 68-94, wherein said at least one initiator is selected from the group consisting of sodium persulfate (NPS), ammonium persulfate (APS), potassium persulfate (KPS) and any combination thereof.

Example 96. The bone cement system according to any one of examples 68-95, wherein said second component comprises at least one co-initiator.

Example 97. The bone cement system according to any one of examples 68-96, wherein said co-initiator is ascorbic acid.

Example 98. The bone cement system according to any one of examples 68-97, wherein said co-initiator is a modified molecule based on ascorbic acid.

Example 99. The bone cement system according to any one of examples 68-98, wherein a ratio of co-initiation to initiation is from about 0.1:1 to about 1:1.

Example 100. The bone cement system according to any one of examples 68-99, wherein said second component comprises at least one stabilizer.

Example 101. The bone cement system according to any one of examples 68-100, wherein said second component comprises at least one additive.

Example 102. The bone cement system according to any one of examples 68-101, wherein said at least one additive is a pharmaceutical.

Example 103. The bone cement system according to any one of examples 68-102, wherein said at least one additive is selected from the group consisting of antibiotics, stem cells, bone growth factors and any combination thereof.

Example 104. The bone cement system according to any one of examples 68-103, wherein a concentration of said initiator is from about 1:10 to about 1:500 in relation to said methacrylic functional groups.

Example 105. The bone cement system according to any one of examples 68-104, wherein a ratio between said first component to said second component is from about 1:10 to about 1:1.

Example 106. The bone cement system according to any one of examples 68-105, wherein said medical grade bone cement, after being mixed, is characterized by having a low viscosity level of from about 10 Pa·s to about 100 Pa·s.

Example 107. The bone cement system according to any one of examples 68-106, wherein said medical grade bone cement, after being mixed, is characterized by having a medium viscosity level of from about 100 Pa·s to about 500 Pa·s.

Example 108. The bone cement system according to any one of examples 68-107, wherein said medical grade bone cement, after being mixed, is characterized by having a high viscosity level of from about 500 Pa·s to about 1500 Pa·s.

Example 109. The bone cement system according to any one of examples 68-108, wherein said medical grade bone cement is characterized by a working time from about 1 min to about 40 min at room temperature.

Example 110. The bone cement system according to any one of examples 68-109, wherein said medical grade bone cement is characterized by a hardening time starting from about an end of said working time to about 24 hours.

Example 111. The bone cement system according to any one of examples 68-110, wherein said medical grade bone cement is characterized by achieving a non-deformable stage after from about 1 minute to about 5 minutes from about an end of said working time.

Example 112. The bone cement system according to any one of examples 68-111, wherein a polymerization process is affected by temperature.

Example 113. The bone cement system according to any one of examples 68-112, wherein a polymerization process is affected by moisture.

Example 114. The bone cement system according to any one of examples 68-113, wherein exposure of said bone cement to temperatures of from about 35° C. to about 40° C. accelerates the polymerization process time by a factor of about 2 to about 5 compared to process time at temperatures of from about 20° C. to about 25° C.

Example 115. The bone cement system according to any one of examples 68-114, wherein exposure of said bone cement to moisture accelerates the polymerization process time by a factor of about 2 to about 5.

Example 116. The bone cement system according to any one of examples 68-115, wherein concomitant exposure of said bone cement to moisture and to temperatures of from about 35° C. to about 40° C. accelerates the polymerization process time by a factor of about 2 to about 25.

Example 117. The bone cement system according to any one of examples 68-116, wherein said medical grade bone cement comprises a color indicator for the polymerization process.

Example 118. The bone cement system according to any one of examples 68-117, wherein said working time is affected by temperature.

Example 119. The bone cement system according to any one of examples 68-118, wherein said working time process is affected by moisture.

Example 120. The bone cement system according to any one of examples 68-119, wherein exposure of said bone cement to temperatures of from about 35° C. to about 40° C. reduces said working time by a factor of about 2 to about 5 compared to said working time at room temperatures of from about 20° C. to about 25° C.

Example 121. The bone cement system according to any one of examples 68-120, wherein exposure of said bone cement to moisture reduces said working time by a factor of about 2 to about 5.

Example 122. The bone cement system according to any one of examples 68-121, wherein concomitant exposure of said bone cement to moisture and to temperatures of from about 35° C. to about 40° C. reduces said working time by a factor of about 2 to about 25.

Example 123. The bone cement system according to any one of examples 68-122, wherein said medical grade bone cement comprises a color indicator for said working time.

Example 124. The bone cement system according to any one of examples 68-123, wherein said first polymer material is hydrophilic at least in part.

Example 125. The bone cement system according to any one of examples 68-124, further comprising a second component comprising a hydrophilic initiator.

Example 126. The bone cement system according to any one of examples 68-125, wherein said first polymer material is hydrophilic only in part.

Example 127. The bone cement system according to any one of examples 68-126, further comprising a second component comprising a hydrophilic initiator.

Example 128. The bone cement system according to any one of examples 68-127, wherein said first polymer material is hygroscopic at least in part.

Example 129. The bone cement system according to any one of examples 68-128, wherein said first polymer material is hygroscopic only in part.

Example 130. The bone cement system according to any one of examples 68-129, wherein said medical grade bone cement is stored at room temperature before being used.

Example 131. The bone cement system according to any one of examples 68-130, wherein said medical grade bone cement comprises a shelf-life of from about 1 year to about 10 years.

Example 132. The bone cement system according to any one of examples 68-131, wherein said components and ingredients thereof are divided into more than two parts before mixing.

Example 133. The bone cement system according to any one of examples 68-132, wherein said bone cement changes color during the polymerization process thereby providing an intrinsic curing indicator.

Example 134. The bone cement system according to any one of examples 68-133, wherein said polymerization process is characterized by being a low exothermal polymerization process.

Example 135. A medical grade bone cement having two mixable viscous components, the bone cement comprising:
a. a first component comprising:
i. a monomer-free PVGMA/PGMAE polymer;
ii. a methacrylated solvent;
b. a second component comprising:
iii. at least one initiator;
iv. at least one polymer non-reactive to said initiator; and
v. ascorbic acid as co-initiator.

Example 136. The medical grade bone cement according to example 135, wherein said methacrylated solvent is di/mono-PEG/PPG.

Example 137. The medical grade bone cement according to example 135 or example 136, wherein said first component and said second component are characterized by having similar viscosities within a factor of 10.

Example 138. The medical grade bone cement according to any one of examples 135-137, wherein said first component is less than 10% by weight of monomers.

Example 139. The medical grade bone cement according to any one of examples 135-138, wherein said first component is monomer-free.

Example 140. The medical grade bone cement according to any one of examples 135-139, wherein said first component comprises at least from about 20% to about 80% by weight of said first polymer.

Example 141. The medical grade bone cement according to any one of examples 135-140, wherein said second component comprises at least from about 40% to about 100% by weight of said second polymer.

Example 142. The medical grade bone cement according to any one of examples 135-141, wherein said second component does not require pre-processing actions before being used.

Example 143. The medical grade bone cement according to any one of examples 135-142, wherein said bone cement is provided as a ready to use bone cement in a dedicated implementation device.

Example 144. The medical grade bone cement according to any one of examples 135-143, wherein said first polymer material comprises a Polyvinyl alcohol (PVA) based polymer modified with methacrylic groups.

Example 145. The medical grade bone cement according to any one of examples 135-144, wherein said first polymer material comprising said PVA based polymer modified with methacrylic groups is PVGMA.

Example 146. The medical grade bone cement according to any one of examples 135-145, wherein said PVA is characterized by a degree of hydrolyzation of from about 40% to about 99.9%.

Example 147. The medical grade bone cement according to any one of examples 135-146, wherein a methacrylate modification along said PVA is characterized by a ratio of from about 1:100 to about 20:1.

Example 148. The medical grade bone cement according to any one of examples 135-147, wherein a methacrylate modification along said PVA is characterized by addition of branches of poly glycidyl methacrylate ether (PGMAE).

Example 149. The medical grade bone cement according to any one of examples 135-148, wherein a minimum quantity of PGMAE in said first polymer material is about 3 times more by weight than said PVA.

Example 150. The medical grade bone cement according to any one of examples 135-149, wherein a minimum quantity of PGMAE in said first polymer material is about 1.2 times larger by molar ratio than said PVA.

Example 151. The medical grade bone cement according to any one of examples 135-150, wherein a minimum quantity of PGMAE in said first polymer material is in a ratio of 1:10 of PGMAE/PVA and said PGMAE comprises a length of 4 units.

Example 152. The medical grade bone cement according to any one of examples 135-151, wherein said first polymer material is 100% PGMAE.

Example 153. The medical grade bone cement according to any one of examples 135-152, wherein said first component comprises a solvent selected from group consisting of poly ethylene glycol di/mono-methacrylate, poly propylene glycol di/mono-methacrylate, poloxamer di/mono-methacrylate and any combination thereof.

Example 154. The medical grade bone cement according to any one of examples 135-153, wherein said first component comprises a PEG/PPG based solvent.

Example 155. The medical grade bone cement according to any one of examples 135-154, wherein said first component comprises a linker selected from the group consisting of Bisphenol A Glycidyl Dimethacrylate (bisGMA), bisphenol A Ethylene Glycol Dimethacrylate (bisEMA), triethylene glycol dimethacrylate (TEGDMA), Urethane Dimethacrylate (UDMA) and any combination thereof.

Example 156. The medical grade bone cement according to any one of examples 135-155, wherein said first component comprises at least one additive selected from the group consisting of a radiopaque material, an inhibitor, water and any combination thereof.

Example 157. The medical grade bone cement according to any one of examples 135-156, wherein said first component is characterized by the ability to absorb water.

Example 158. The medical grade bone cement according to any one of examples 135-157, wherein ether groups between said methacrylic functional groups provide said ability to absorb water.

Example 159. The medical grade bone cement according to any one of examples 135-158, wherein said PVA provide said ability to absorb water.

Example 160. The medical grade bone cement according to any one of examples 135-159, wherein said second polymer is selected from the group consisting of Pluronic P123, Pluronic P105, Pluronic P85, paste PEG1000, paste PPG8000, mixture of short liquid and long solid PEGs PPGs, poloxamers and any combination thereof.

Example 161. The medical grade bone cement according to any one of examples 135-160, wherein said second polymer is a bio-degradable polymer.

Example 162. The medical grade bone cement according to any one of examples 135-161, wherein said bio-degradable polymer are one or more of polyesters and polysaccharides.

Example 163. The medical grade bone cement according to any one of examples 135-162, wherein said at least one initiator is selected from the group consisting of sodium persulfate (NPS), ammonium persulfate (APS), potassium persulfate (KPS) and any combination thereof.

Example 164. The medical grade bone cement according to any one of examples 135-163, wherein said second component comprises at least one co-initiator.

Example 165. The medical grade bone cement according to any one of examples 135-164, wherein said co-initiator is ascorbic acid.

Example 166. The medical grade bone cement according to any one of examples 135-165, wherein said co-initiator is a modified molecule based on ascorbic acid.

Example 167. The medical grade bone cement according to any one of examples 135-166, wherein a ratio of co-initiation to initiation is from about 0.1:1 to about 1:1.

Example 168. The medical grade bone cement according to any one of examples 135-167, wherein said second component comprises at least one stabilizer.

Example 169. The medical grade bone cement according to any one of examples 135-168, wherein said second component comprises at least one additive.

Example 170. The medical grade bone cement according to any one of examples 135-169, wherein said at least one additive is a pharmaceutical.

Example 171. The medical grade bone cement according to any one of examples 135-170, wherein said at least one additive is selected from the group consisting of antibiotics, stem cells, bone growth factors and any combination thereof.

Example 172. The medical grade bone cement according to any one of examples 135-171, wherein a concentration of said initiator is from about 1:10 to about 1:500 in relation to said methacrylic functional groups.

Example 173. The medical grade bone cement according to any one of examples 135-172, wherein a ratio between said first component and said second component is from about 1:10 to about 1:1.

Example 174. The medical grade bone cement according to any one of examples 135-173, wherein said medical grade bone cement, after being mixed, is characterized by having a low viscosity level of from about 10 Pa·s to about 100 Pa·s.

Example 175. The medical grade bone cement according to any one of examples 135-174, wherein said medical grade bone cement, after being mixed, is characterized by having a medium viscosity level of from about 100 Pa·s to about 500 Pa·s.

Example 176. The medical grade bone cement according to any one of examples 135-175, wherein said medical grade bone cement, after being mixed, is characterized by having a high viscosity level of from about 500 Pa·s to about 1500 Pa·s.

Example 177. The medical grade bone cement according to any one of examples 135-176, wherein said medical grade bone cement is characterized by a working time from about 1 min to about 40 min at room temperature.

Example 178. The medical grade bone cement according to any one of examples 135-177, wherein said medical grade bone cement is characterized by a hardening time starting from about an end of said working time to about 24 hours.

Example 179. The medical grade bone cement according to any one of examples 135-178, wherein said medical grade bone cement is characterized by achieving a non-deformable stage after from about 1 minute to about 5 minutes from about an end of said working time.

Example 180. The medical grade bone cement according to any one of examples 135-179, wherein a polymerization process is affected by temperature.

Example 181. The medical grade bone cement according to any one of examples 135-180, wherein a polymerization process is affected by moisture.

Example 182. The medical grade bone cement according to any one of examples 135-181, wherein exposure of said bone cement to temperatures of from about 35° C. to about 40° C. accelerates the polymerization process from about 2 times to about 5 times compared to process time at temperatures of from about 20° C. to about 25° C.

Example 183. The medical grade bone cement according to any one of examples 135-182, wherein exposure of said bone cement to moisture accelerates the polymerization process from about 2 times to about 5 times.

Example 184. The medical grade bone cement according to any one of examples 135-183, wherein concomitant exposure of said bone cement to moisture and to temperatures of from about 35° C. to about 40° C. accelerates the polymerization process from about 2 times to about 25 times.

Example 185. The medical grade bone cement according to any one of examples 135-184, wherein said medical grade bone cement comprises a color indicator for the polymerization process.

Example 186. The medical grade bone cement according to any one of examples 135-185, wherein said working time is affected by temperature.

Example 187. The medical grade bone cement according to any one of examples 135-186, wherein said working time process is affected by moisture.

Example 188. The medical grade bone cement according to any one of examples 135-187, wherein exposure of said bone cement to temperatures of from about 35° C. to about 40° C. reduces said working time from about 2 times to about 5 times compared to said working time at room temperatures of from about 20° C. to about 25° C.

Example 189. The medical grade bone cement according to any one of examples 135-188, wherein exposure of said bone cement to moisture reduces said working time from about 2 times to about 5 times.

Example 190. The medical grade bone cement according to any one of examples 135-189, wherein concomitant exposure of said bone cement to moisture and to temperatures of from about 35° C. to about 40° C. reduces said working time from about 2 times to about 25 times.

Example 191. The medical grade bone cement according to any one of examples 135-190, wherein said medical grade bone cement comprises a color indicator for said working time.

Example 192. The medical grade bone cement according to any one of examples 135-191, wherein said first polymer material is hydrophilic at least in part.

Example 193. The medical grade bone cement according to any one of examples 135-192, further comprising a second component comprising a hydrophilic initiator.

Example 194. The medical grade bone cement according to any one of examples 135-193, wherein said first polymer material is hydrophilic only in part.

Example 195. The medical grade bone cement according to any one of examples 135-194, further comprising a second component comprising a hydrophilic initiator.

Example 196. The medical grade bone cement according to any one of examples 135-195, wherein said first polymer material is hygroscopic at least in part.

Example 197. The medical grade bone cement according to any one of examples 135-196, wherein said first polymer material is hygroscopic only in part.

Example 198. The medical grade bone cement according to any one of examples 135-197, wherein said medical grade bone cement is stored at room temperature before being used.

Example 199. The medical grade bone cement according to any one of examples 135-198, wherein said medical grade bone cement comprises a shelf-life of from about 1 year to about 10 years.

Example 200. The medical grade bone cement according to any one of examples 135-199, wherein said components and ingredients thereof are divided into more than two parts before mixing.

Example 201. The medical grade bone cement according to any one of examples 135-200, wherein said bone cement changes color during polymerization process thereby providing an intrinsic curing indicator.

Example 202. The medical grade bone cement according to any one of examples 135-201, wherein said polymerization process is characterized by being a low exothermal polymerization process.

Example 203. A bone cement comprising two mixable components, wherein said two mixable components are characterized by having similar viscosities within a factor of 10.

Example 204. A bone cement comprising two mixable components, wherein a first component is hydrophilic at least in part and a second component comprises at least one initiator in a hydrophilic base.

Example 205. A bone cement comprising two mixable components, wherein a first component comprises a first polymer and a second component comprises a second polymer in paste form that acts as a carrier for an initiator for said first polymer; said second polymer does not react with said initiator.

Example 206. A bone cement comprising two mixable components, wherein a first component comprises a first polymer and a second component comprises a second polymer that acts as a carrier for an initiator for said first polymer; said second polymer does not react with said initiator; and wherein said initiator are persulfate salts which are used with ascorbic acid as co-initiators.

Example 207. A bone cement comprising two mixable components, wherein a first component comprises a first polymer comprising a Polyvinyl alcohol (PVA) based polymer modified by addition of branches of poly glycidyl methacrylate ether (PGMAE) and a PEG and/or PPG based solvent; and wherein a second component comprises a second polymer that acts as a carrier for an initiator for said first polymer; said second polymer does not react with said initiator; and wherein said initiator are persulfate salts which are used with ascorbic acid or a modified molecule based on ascorbic acid as co-initiators.

Example 208. The medical grade bone cement according to any one of examples 203 or example 204 or example 205 or example 206 or example 207, wherein said first component and said second component are characterized by having similar viscosities within a factor of 10.

Example 209. The medical grade bone cement according to any one of examples 203 or example 204 or example 205 or example 206 or example 207 and example 208, wherein said first component is less than 10% by weight of monomers.

Example 210. The medical grade bone cement according to any one of examples 203 or example 204 or example 205 or example 206 or example 207 and any one of examples 208-209, wherein said first component is monomer-free.

Example 211. The medical grade bone cement according to any one of examples 203 or example 204 or example 205 or example 206 or example 207 and any one of examples 208-210, wherein said first component comprises at least from about 20% to about 80% by weight of said first polymer.

Example 212. The medical grade bone cement according to any one of examples 203 or example 204 or example 205 or example 206 or example 207 and any one of examples 208-211, wherein said second component comprises at least from about 40% to about 100% by weight of said second polymer.

Example 213. The medical grade bone cement according to any one of examples 203 or example 204 or example 205 or example 206 or example 207 and any one of examples 208-212, wherein said second component does not require pre-processing actions before being used.

Example 214. The medical grade bone cement according to any one of examples 203 or example 204 or example 205 or example 206 or example 207 and any one of examples 208-213, wherein said bone cement is provided as a ready to use bone cement in a dedicated implementation device.

Example 215. The medical grade bone cement according to any one of examples 203 or example 204 or example 205 or example 206 or example 207 and any one of examples 208-214, wherein said first polymer material comprises a Polyvinyl alcohol (PVA) based polymer modified with methacrylic groups.

Example 216. The medical grade bone cement according to any one of examples 203 or example 204 or example 205 or example 206 or example 207 and any one of examples 208-215, wherein said first polymer material comprising said PVA based polymer modified with methacrylic groups is PVGMA.

Example 217. The medical grade bone cement according to any one of examples 203 or example 204 or example 205 or example 206 or example 207 and any one of examples 208-216, wherein said PVA is characterized by a degree of hydrolyzation of from about 40% to about 99.9%.

Example 218. The medical grade bone cement according to any one of examples 203 or example 204 or example 205 or example 206 or example 207 and any one of examples 208-217, wherein a methacrylate modification along said PVA is characterized by a ratio of from about 1:100 to about 20:1.

Example 219. The medical grade bone cement according to any one of examples 203 or example 204 or example 205 or example 206 or example 207 and any one of examples 208-218, wherein a methacrylate modification along said PVA is characterized by addition of branches of poly glycidyl methacrylate ether (PGMAE).

Example 220. The medical grade bone cement according to any one of examples 203 or example 204 or example 205 or example 206 or example 207 and any one of examples 208-219, wherein a minimum quantity of PGMAE in said first polymer material is about 3 times more by weight than said PVA.

Example 221. The medical grade bone cement according to any one of examples 203 or example 204 or example 205 or example 206 or example 207 and any one of examples 208-220, wherein a minimum quantity of PGMAE in said first polymer material is about 1.2 times larger by molar ratio than said PVA.

Example 222. The medical grade bone cement according to any one of examples 203 or example 204 or example 205 or example 206 or example 207 and any one of examples 208-221, wherein a minimum quantity of PGMAE in said first polymer material is in a ratio of 1:10 of PGMAE/PVA and said PGMAE comprises a length of 4 units.

Example 223. The medical grade bone cement according to any one of examples 203 or example 204 or example 205 or example 206 or example 207 and any one of examples 208-222, wherein said first polymer material is 100% PGMAE.

Example 224. The medical grade bone cement according to any one of examples 203 or example 204 or example 205 or example 206 or example 207 and any one of examples 208-223, wherein said first component comprises a solvent selected from group consisting of poly ethylene glycol di/mono-methacrylate, poly propylene glycol di/mono-methacrylate, poloxamer di/mono-methacrylate and any combination thereof.

Example 225. The medical grade bone cement according to any one of examples 203 or example 204 or example 205 or example 206 or example 207 and any one of examples 208-224, wherein said first component comprises a PEG/PPG based solvent.

Example 226. The medical grade bone cement according to any one of examples 203 or example 204 or example 205 or example 206 or example 207 and any one of examples 208-225, wherein said first component comprises a linker selected from the group consisting of Bisphenol A Glycidyl Dimethacrylate (bisGMA), bisphenol A Ethylene Glycol Dimethacrylate (bisEMA), triethylene glycol dimethacrylate (TEGDMA), Urethane Dimethacrylate (UDMA) and any combination thereof.

Example 227. The medical grade bone cement according to any one of examples 203 or example 204 or example 205 or example 206 or example 207 and any one of examples 208-226, wherein said first component comprises at least one additive selected from the group consisting of a radiopaque material, an inhibitor, water and any combination thereof.

Example 228. The medical grade bone cement according to any one of examples 203 or example 204 or example 205 or example 206 or example 207 and any one of examples 208-227, wherein said first component is characterized by the ability to absorb water.

Example 229. The medical grade bone cement according to any one of examples 203 or example 204 or example 205 or example 206 or example 207 and any one of examples 208-228, wherein ether groups between said methacrylic functional groups provide said ability to absorb water.

Example 230. The medical grade bone cement according to any one of examples 203 or example 204 or example 205 or example 206 or example 207 and any one of examples 208-229, wherein said PVA provide said ability to absorb water.

Example 231. The medical grade bone cement according to any one of examples 203 or example 204 or example 205 or example 206 or example 207 and any one of examples 208-230, wherein said second polymer is selected from the group consisting of Pluronic P123, Pluronic P105, Pluronic P85, paste PEG1000, paste PPG8000, mixture of short liquid and long solid PEGs PPGs, poloxamers and any combination thereof.

Example 232. The medical grade bone cement according to any one of examples 203 or example 204 or example 205 or example 206 or example 207 and any one of examples 208-231, wherein said second polymer is a bio-degradable polymer.

Example 233. The medical grade bone cement according to any one of examples 203 or example 204 or example 205 or example 206 or example 207 and any one of examples 208-232, wherein said bio-degradable polymer are one or more of polyesters and polysaccharides.

Example 234. The medical grade bone cement according to any one of examples 203 or example 204 or example 205 or example 206 or example 207 and any one of examples 208-233, wherein said at least one initiator is selected from the group consisting of sodium persulfate (NPS), ammonium persulfate (APS), potassium persulfate (KPS) and any combination thereof.

Example 235. The medical grade bone cement according to any one of examples 203 or example 204 or example 205 or example 206 or example 207 and any one of examples 208-234, wherein said second component comprises at least one co-initiator.

Example 236. The medical grade bone cement according to any one of examples 203 or example 204 or example 205 or example 206 or example 207 and any one of examples 208-235, wherein said co-initiator is ascorbic acid.

Example 237. The medical grade bone cement according to any one of examples 203 or example 204 or example 205 or example 206 or example 207 and any one of examples 208-236, wherein said co-initiator is a modified molecule based on ascorbic acid.

Example 238. The medical grade bone cement according to any one of examples 203 or example 204 or example 205 or example 206 or example 207 and any one of examples 208-237, wherein a ratio of co-initiation to initiation is from about 0.1:1 to about 1:1.

Example 239. The medical grade bone cement according to any one of examples 203 or example 204 or example 205 or example 206 or example 207 and any one of examples 208-238, wherein said second component comprises at least one stabilizer.

Example 240. The medical grade bone cement according to any one of examples 203 or example 204 or example 205 or example 206 or example 207 and any one of examples 208-239, wherein said second component comprises at least one additive.

Example 241. The medical grade bone cement according to any one of examples 203 or example 204 or example 205 or example 206 or example 207 and any one of examples 208-240, wherein said at least one additive is a pharmaceutical.

Example 242. The medical grade bone cement according to any one of examples 203 or example 204 or example 205 or example 206 or example 207 and any one of examples 208-241, wherein said at least one additive is selected from the group consisting of antibiotics, stem cells, bone growth factors and any combination thereof.

Example 243. The medical grade bone cement according to any one of examples 203 or example 204 or example 205 or example 206 or example 207 and any one of examples 208-242, wherein a concentration of said initiator is from about 1:10 to about 1:500 in relation to said methacrylic functional groups.

Example 244. The medical grade bone cement according to any one of examples 203 or example 204 or example 205 or example 206 or example 207 and any one of examples 208-243, wherein a ratio between said first component to said second component is from about 1:10 to about 1:1.

Example 245. The medical grade bone cement according to any one of examples 203 or example 204 or example 205 or example 206 or example 207 and any one of examples 208-244, wherein said medical grade bone cement, after being mixed, is characterized by having a low viscosity level of from about 10 Pa·s to about 100 Pa·s.

Example 246. The medical grade bone cement according to any one of examples 203 or example 204 or example 205 or example 206 or example 207 and any one of examples 208-245, wherein said medical grade bone cement, after being mixed, is characterized by having a medium viscosity level of from about 100 Pa·s to about 500 Pa·s.

Example 247. The medical grade bone cement according to any one of examples 203 or example 204 or example 205 or example 206 or example 207 and any one of examples 208-246, wherein said medical grade bone cement, after being mixed, is characterized by having a high viscosity level of from about 500 Pa·s to about 1500 Pa·s.

Example 248. The medical grade bone cement according to any one of examples 203 or example 204 or example 205 or example 206 or example 207 and any one of examples 208-247, wherein said medical grade bone cement is characterized by a working time from about 1 min to about 40 min at room temperature.

Example 249. The medical grade bone cement according to any one of examples 203 or example 204 or example 205 or example 206 or example 207 and any one of examples 208-248, wherein said medical grade bone cement is characterized by a hardening time starting from about an end of said working time to about 24 hours.

Example 250. The medical grade bone cement according to any one of examples 203 or example 204 or example 205 or example 206 or example 207 and any one of examples 208-249, wherein said medical grade bone cement is characterized by achieving a non-deformable stage after from about 1 minute to about 5 minutes from about an end of said working time.

Example 251. The medical grade bone cement according to any one of examples 203 or example 204 or example 205 or example 206 or example 207 and any one of examples 208-250, wherein a polymerization process is affected by temperature.

Example 252. The medical grade bone cement according to any one of examples 203 or example 204 or example 205 or example 206 or example 207 and any one of examples 208-251, wherein a polymerization process is affected by moisture.

Example 253. The medical grade bone cement according to any one of examples 203 or example 204 or example 205 or example 206 or example 207 and any one of examples 208-252, wherein exposure of said bone cement to temperatures of from about 35° C. to about 40° C. accelerates the polymerization process from about 2 times to about 5 times compared to process time at temperatures of from about 20° C. to about 25° C.

Example 254. The medical grade bone cement according to any one of examples 203 or example 204 or example 205 or example 206 or example 207 and any one of examples 208-253, wherein exposure of said bone cement to moisture accelerates the polymerization process from about 2 times to about 5 times.

Example 255. The medical grade bone cement according to any one of examples 203 or example 204 or example 205 or example 206 or example 207 and any one of examples 208-254, wherein concomitant exposure of said bone cement to moisture and to temperatures of from about 35° C. to about 40° C. accelerates the polymerization process from about 2 times to about 25 times.

Example 256. The medical grade bone cement according to any one of examples 203 or example 204 or example 205 or example 206 or example 207 and any one of examples 208-255, wherein said medical grade bone cement comprises a color indicator for the polymerization process.

Example 257. The medical grade bone cement according to any one of examples 203 or example 204 or example 205 or example 206 or example 207 and any one of examples 208-256, wherein said working time is affected by temperature.

Example 258. The medical grade bone cement according to any one of examples 203 or example 204 or example 205 or example 206 or example 207 and any one of examples 208-257, wherein said working time process is affected by moisture.

Example 259. The medical grade bone cement according to any one of examples 203 or example 204 or example 205 or example 206 or example 207 and any one of examples 208-258, wherein exposure of said bone cement to temperatures of from about 35° C. to about 40° C. reduces said working time from about 2 times to about 5 times compared to said working time at room temperatures of from about 20° C. to about 25° C.

Example 260. The medical grade bone cement according to any one of examples 203 or example 204 or example 205 or example 206 or example 207 and any one of examples 208-259, wherein exposure of said bone cement to moisture reduces said working time from about 2 times to about 5 times.

Example 261. The medical grade bone cement according to any one of examples 203 or example 204 or example 205 or example 206 or example 207 and any one of examples 208-260, wherein concomitant exposure of said bone cement to moisture and to temperatures of from about 35° C. to about 40° C. reduces said working time from about 2 times to about 25 times.

Example 262. The medical grade bone cement according to any one of examples 203 or example 204 or example 205 or example 206 or example 207 and any one of examples 208-261, wherein said medical grade bone cement comprises a color indicator for said working time.

Example 263. The medical grade bone cement according to any one of examples 203 or example 204 or example 205 or example 206 or example 207 and any one of examples 208-262, wherein said first polymer material is hydrophilic at least in part.

Example 264. The medical grade bone cement according to any one of examples 203 or example 204 or example 205 or example 206 or example 207 and any one of examples 208-263, further comprising a second component comprising a hydrophilic initiator.

Example 265. The medical grade bone cement according to any one of examples 203 or example 204 or example 205 or example 206 or example 207 and any one of examples 208-264, wherein said first polymer material is hydrophilic only in part.

Example 266. The medical grade bone cement according to any one of examples 203 or example 204 or example 205 or example 206 or example 207 and any one of examples 208-265, further comprising a second component comprising a hydrophilic initiator.

Example 267. The medical grade bone cement according to any one of examples 203 or example 204 or example 205 or example 206 or example 207 and any one of examples 208-266, wherein said first polymer material is hygroscopic at least in part.

Example 268. The medical grade bone cement according to any one of examples 203 or example 204 or example 205 or example 206 or example 207 and any one of examples 208-267, wherein said first polymer material is hygroscopic only in part.

Example 269. The medical grade bone cement according to any one of examples 203 or example 204 or example 205 or example 206 or example 207 and any one of examples 208-268, wherein said medical grade bone cement is stored at room temperature before being used.

Example 270. The medical grade bone cement according to any one of examples 203 or example 204 or example 205 or example 206 or example 207 and any one of examples 208-269, wherein said medical grade bone cement comprises a shelf-life of from about 1 year to about 10 years.

Example 271. The medical grade bone cement according to any one of examples 203 or example 204 or example 205 or example 206 or example 207 and any one of examples 208-270, wherein said components and ingredients thereof are divided into more than two parts before mixing.

Example 272. The medical grade bone cement according to any one of examples 203 or example 204 or example 205 or example 206 or example 207 and any one of examples 208-271, wherein said bone cement changes color during polymerization process thereby providing an intrinsic curing indicator.

Example 273. The medical grade bone cement according to any one of examples 203 or example 204 or example 205 or example 206 or example 207 and any one of examples 208-272, wherein said polymerization process is characterized by being a low exothermal polymerization process.

Example 274. A method to treat a tissue of a patient, comprising:
a. providing at least two components in separate containers;
b. mixing said at least two components in a mixing unit, thereby generating a mixed component;
c. delivering said mixed component to the targeted tissue; and
d. stimulating said at least one implant to accelerate cross-linking of said mixed component from a first configuration to a second harder configuration;
wherein said stimulating is performed by the natural heat and/or the moisture of the body of said patient.

Example 275. Use of a material for medical grade implants comprising a first polymer comprising at least 20% by weight of a Polyvinyl alcohol (PVA) based polymer modified by addition of branches of poly glycidyl methacrylate ether (PGMAE), at least one initiator selected to cause cross-linking of said first polymer; said at least one initiator carried in at least 50% by weight of a second polymer material that does not react with said initiator; said second component being a paste.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an image of an exemplary implementation device, according to some embodiments of the invention;

FIG. 2 is an image of an exemplary implementation device mounted and ready to be used, according to some embodiments of the invention;

FIG. 3 is an image of an exemplary implementation device being used and the two compounds being mixed inside the mixing unit by being forced to pass through the sequence of mixing levels, according to some embodiments of the invention;

FIG. 4 is an image of an exemplary mixed cement being applied from an exemplary implementation device, according to some embodiments of the invention;

FIG. 5 are exemplary molecules of Component A and Component B, according to some embodiments of the invention;

FIG. 6 is an exemplary schematic chain reaction of addition of molecules, according to some embodiments of the invention; and

FIGS. 7a-b are schematic representations of an exemplary_Implementation/Delivery Device according to some embodiments of the invention.

### DESCRIPTION OF SPECIFIC EMBODIMENTS OF THE INVENTION

The present invention, in some embodiments thereof, relates to medical grade and non-medical grade materials, which are configured to change their property following stimulation.

### Overview

An aspect of some embodiments of the invention relates to a monomer free, ready to use, methacrylic bone cement. In some embodiments, the cement is composed of two paste components, which are mixed only during injection when the user proactively mixes them. In some embodiments, the mixed viscose matter has a constant viscosity and is transferred through a narrow opening to fill a void. In some embodiments, after few minutes in site, the viscose matter turns into stiff and stable matter. In some embodiments, the bone cement comprises a PVGMA/PGMAE polymer. In some embodiments, the bone cement is generated by the combination of two components, the first being a cross-linkable polymer and di-methacrylates, the second being a non-covalently binding (non-reacting to the at least one initiator) polymer carrying at least one initiator and optionally at least one co-initiator. In some embodiments, the at least one co-initiator is ascorbic acid. In some embodiments, using non-reacting polymer as fillers improves the mechanical properties of the bone cement. In some embodiments, the non-reacting polymer is used as a carrier for the initiator molecules and optionally also for additives (for example pharmaceuticals, cells, etc.) In some embodiments, the at least one initiator is persulfate salts, optionally used with co-initiators, for example ascorbic acid.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In some embodiments, “medical grade” refers to a material which is within the standards of the different countries, international associations, etc. (e.g. FDA, ASTM, ISO, etc.).

In some embodiments, the terms “implant,” “implants,” “medical grade implants” and “medical implant” are interchangeable. In some embodiments, the terms “delivery device,” “implementation device” are interchangeable.

### Exemplary Bone Cement

In some embodiments, the exemplary bone cement is a ready-to-use bone cement, i.e., the user does not need to perform any pre-mixing actions of the materials, as usually happens with bone cements that come in powder form. In some embodiments, a potential advantage of a ready-to-use bone cement is that it saves time for the surgeon performing the intervention, which also saves money for the hospitals, and potentially avoids human errors in the preparation of the bone cement, which can potentially avoid further interventions and even save lives.

In some embodiments, the bone cement is a monomer free, ready to use methacrylic cement. In some embodiments, the ingredients of the bone cement are divided into two components, which are mixed in an implementation device during the use. In some embodiments, the ingredients of the bone cement are divided into more than two components, which are then mixed in an implementation device. In order to allow a person skilled in the art to understand the invention, two components will be explained. It should be understood that the ingredients of the bone cement with/without additives can be divided into more than two components, for example 3 or 4 or more, and those embodiments are also included in the scope of the invention.

In some embodiments, different ratios of the two components are used to achieve different materials having different levels of elasticity.

| | | Final level of compression |
|---|---|---|
| % of Component A | % of Component B | elasticity (GPa) |
| 90 | 10 | 1-2 |
| 80 | 20 | 0.5-1 |
| 50 | 50 | 0.2-0.5 |
| 30 | 70 | 0.1-0.2 |

In some embodiments, once the two components are mixed, the mixed viscous cement flows out the implementation device with a constant viscosity.

In some embodiments, the viscosity of the mixed material is adjusted to reach the desired values. In some embodiments, when a low viscosity cement is desired it is characterized by a viscosity value, for example, of from about 10 Pa·s to about 100 Pa·s. In some embodiments, when a medium viscosity cement is desired it is characterized by a viscosity value, for example, of from about 100 Pa·s to about 500 Pa·s. In some embodiments, when a high viscosity cement is desired it is characterized by a viscosity value, for example, of from about 500 Pa·s to about 1500 Pa·s.

In some embodiments, the polymerization process begins once the two (or more) components are mixed inside the implementation device. In some embodiments, the polymerization process starts after few minutes, for example, the polymerization process begins after from about 2 minutes to about 4 minutes, optionally after from about 1 minute to about 5 minutes, optionally after from about 30 seconds to about 10 minutes. In some embodiments, the polymerization process is affected by one or more of temperature and moisture. In some embodiments, the polymerization process is accelerated inside the body as the mixed material is exposed to higher temperatures and/or to an increase in moisture (for example liquids of the body). In some embodiments, exposure to body temperatures (from about 35° C. to about 40° C.) accelerates the polymerization process time by a factor of about 2 to about 5 compared to polymerization process time at room temperature (from about 20° C. to about 25° C.), optionally by a factor or about 1.5 to about 8, optionally by a factor of about 1.2 to about 10. In some embodiments, an increase in the exposure to moisture accelerates the polymerization process time by a factor of about 2 to about 5, optionally by a factor of about 1.5 to about 8, optionally by a factor of about 1.2 to about 10. In some embodiments, a concomitant increase in the temperature and in the exposure to moisture accelerates the polymerization process time by a factor of about 2 to about 10, optionally by a factor of about 1.5 to about 20, optionally by a factor of about 1.2 to about 50.

In some embodiments the final compression strength of hardened cured matter is from about 100 MPs to about 150 MPs, and its compression modulus is from about 200 MPa to about 2000 MPa.

In the field of bone cements it is customary to provide “mixing time” (the time for the materials to arrive at a best working viscosity), “working time” (the time from the moment the materials arrive at a best working viscosity until the bone cement is not workable anymore, i.e., is hard) and “hardening time” (the time from when the bone cement is not modifiable anymore until it is completely hard) to each specific bone cement. In some embodiments, as mentioned before, a potential advantage of the cement of the present invention is that the mixing time is almost completely avoided since two (or more) components are provided ready to use in separated cartridges/containers and they are mixed in the implementation device seconds before being implanted in the patient.

In some embodiments, once the materials are mixed in the implementation device the working time of the bone cement begins and it lasts, for example, for a period of time from about 2 minutes to about 10 minutes, optionally from about 1 minute to about 30 minutes, optionally from about 30 seconds to about 40 minutes. In some embodiments, the working time is affected by one or more of temperature and moisture. In some embodiments, the working time is reduced inside the body as the mixed material is exposed to higher temperatures and/or an increase in exposure to moisture (for example liquids of the body) than were present before being injected into the body.

In some embodiments, exposure to body temperatures (from about 35° C. to about 40° C.) reduces the working time by a factor of about 2 to about 5 compared to working time at room temperature (from about 20° C. to about 25° C.), optionally by a factor of about 1.5 to about 8, optionally by a factor of about 1.2 to about 10. In some embodiments, an increase in the exposure to moisture reduces the working time by a factor of about 2 to about 5, optionally by a factor of about 1.5 to about 8, optionally by a factor of about 1.2 to about 10. In some embodiments, a concomitant exposure to higher temperatures (body temperatures) and exposure to moisture (body fluids) reduces the working time by a factor of about 2 to about 10, optionally by a factor of about 1.5 to about 25, optionally by a factor of about 1.2 to about 50.

In some embodiments, storage of the bone cement is as separated components stored in cartridges/containers. In some embodiments, the bone cement, in its separated, ready to use components, is stored at room temperature until it is required to be used. In some embodiments, the bone cement, in its separated, ready to use components, does not require storage in cold temperatures until it is required to be used, for example it is not required to be stored at 4° C. or at −20° C. In some embodiments, the bone cement comprises a shelf life of from about 1 year to about 10 years.

### Exemplary Implementation/Delivery Device

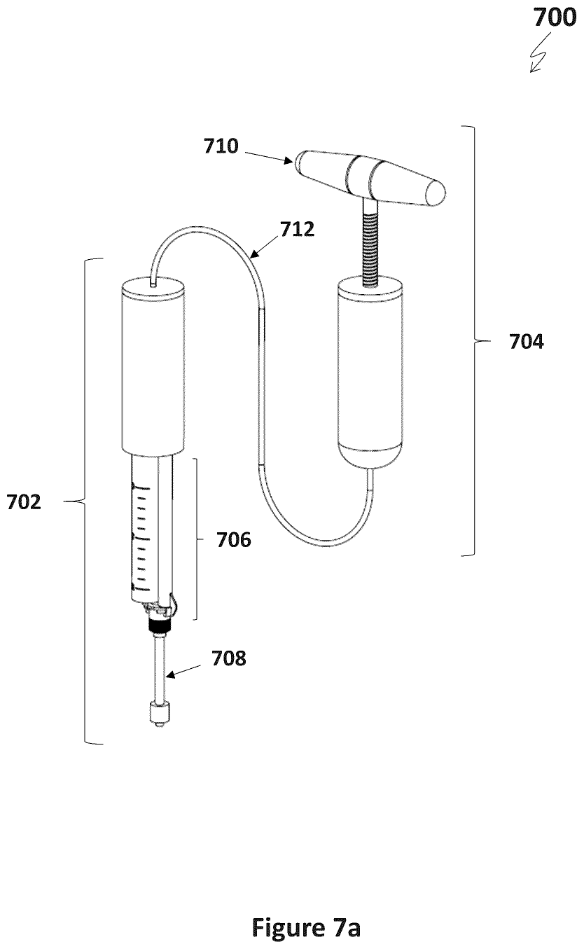

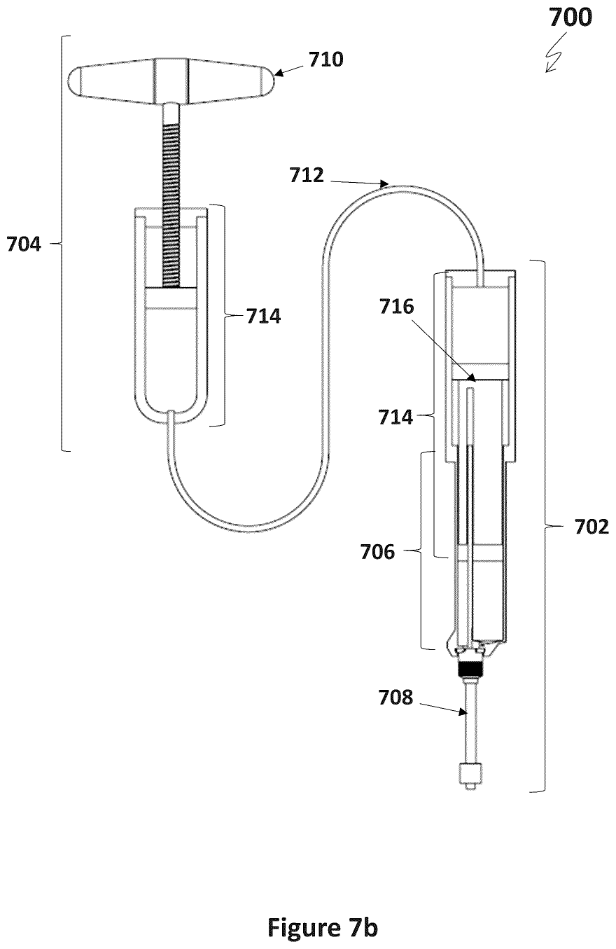

Referring now to FIGS. 7a-b, showing schematic representations of an exemplary Implementation/Delivery Device 700 according to some embodiments of the invention. In some embodiments, the bone cement is supplied in a two-chambered cartridge 706 pre-filled with the two compounds of the bone cement (when the ingredients are divided into two components). In some embodiments, the implementation/delivery device 700 further comprises a disposable mixing unit 708, attachable at the outlet of the two-chambered cartridge 706, where the mixing of the two compounds occurs. In some embodiments, in the disposable mixing unit the polymerization process is initialized. In some embodiments, each compound in the two-chambered cartridge remains unchanged, i.e., the compounds do not polymerize, until they are mixed in the disposable mixing unit. In some embodiments, the mixing unit can be easily replaced at any stage of the implantation procedure. In some embodiments, a potential advantage of having an easily replaceable disposable mixing unit is that it potentially allows for continuous mixing of additional cement for an unlimited amount of time, thereby utilizing any desired amount of bone cement. Additionally, it allows the surgeon to stop the work at any time, replace the mixing unit, and continue or begin a new procedure without losing bone cement material.

In some embodiments, the disposable mixing unit 708 ensures the mixing of the two components before they are delivered to the desired location.

In some embodiments, the mixing unit 708 comprises a sequence of mixing levels. In some embodiments, the number of levels in the sequence, in view of the viscosity of the materials being mixed, determines the quality of the mixing. In some embodiments, for example, a 15-level mixer is used to ensure a high quality of mixing. In some embodiments, in addition, the two mixed components are provided with different colors. In some embodiments, a potential advantage of providing the two components with different colors is that when they mix, a color change is obtained, thereby providing a visual indication of the mixing quality.

In some embodiments, the implementation/delivery device comprises two distinct units 702/704. In some embodiments, the implementation/delivery device comprises a mixing/injection unit 702 and a control unit 704. In some embodiments, the mixing/injection unit comprises the two-chambered cartridge 706 pre-filled with the two compounds of the bone cement and the mixer 708. In some embodiments, in the mixing/injection unit 702, as it name states, the mixing of the bone is performed, optionally while being injected into the patient. In some embodiments, the control unit 704 comprises an actuator 710 (in the form of a handle or a button), which controls (start/stop) the mixing injection unit 702. In some embodiments, the control unit 704 is configured to control the flow in which the bone cement is delivered. In some embodiments, the mixing/injection unit 702 and a control unit 704 are separated one from another, where the mixing/injection unit 702 is usually located distally from the user, which is in the vicinity of the patient, while the control unit 704 is located proximally the user. In some embodiments, the mixing/injection unit 702 and a control unit 704 are connected to each other by one or more connectors 712 configured to transmit the activation/deactivation commands. In some embodiments, the implementation/delivery device 700 utilizes one or more of hydraulic, mechanical and electrical actuation mechanisms 714 (in FIGS. 7a-b a hydraulic mechanism is shown). In some embodiments, the actuation mechanism 714 is configured to translate a gentle and easy action performed in the control unit 704 into a high-intensity action, which optionally requires high forces, in the mixing/injection unit 702. For example, in FIG. 7b a hydraulic mechanism 714 is shown, where rotation of the actuator 710 causes hydraulic liquid to move from the control unit 704 over the connector 712 into the mixing/injection unit 702, where, in this example, the hydraulic liquid pushes (or pulls) a dedicated plunger 716 that causes measured amounts of material from each of the chambers 706 into the mixer 708. In some embodiments, movement of the plunger is always the same in the two chambers, in the meaning that, the plunger is configured to move a same distance in both (or all) barrels. In some embodiments, the plunger is not configured to move different distances in different barrels. In some embodiments, a potential advantage of having a two-part implementation/delivery device is that it allows the user to separate between the part that delivers the bone cement and the actuation part, thereby potentially reducing the chances of mistakenly moving the device while the bone cement is being delivered. In some embodiments, additionally, another potential advantage is that it cases the manipulation of the device by removing the need from the user to apply direct high forces on the mixing/injection unit while delivering the bone cement. Since often the bone cements are very viscous, the user is forced to apply high forces to cause the bone cements parts to exit the barrel and get mixed in the mixer. In some embodiments, this is avoid by the used of one or more of hydraulic, mechanical and electrical actuation mechanisms, as disclosed herein.

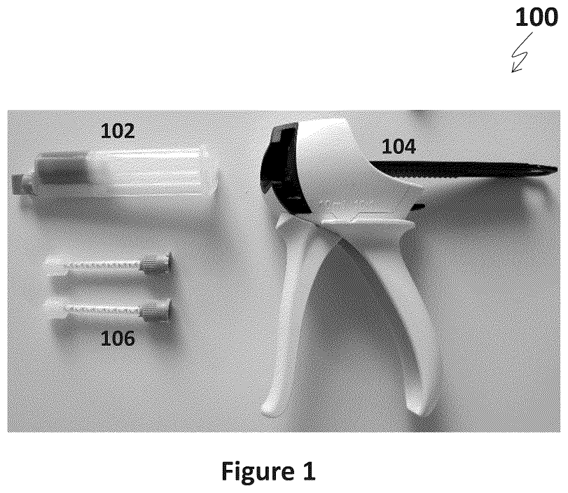

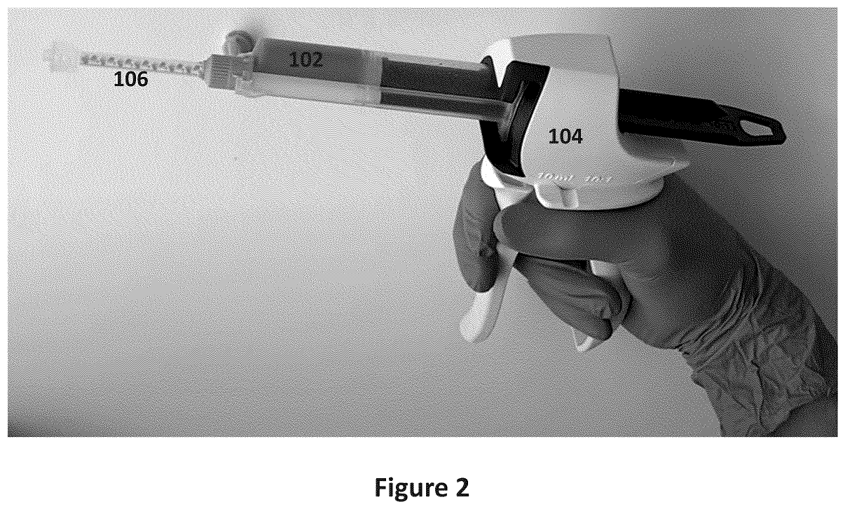

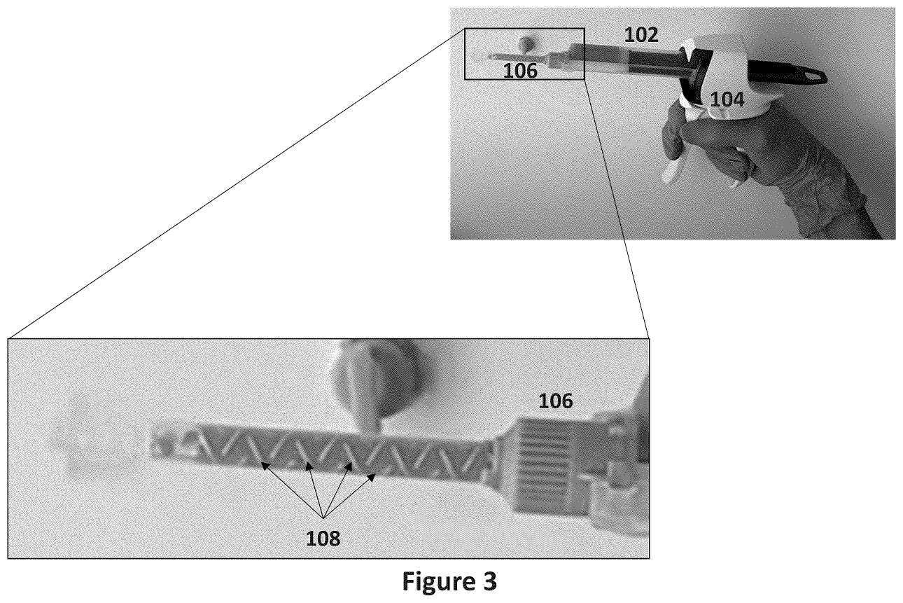

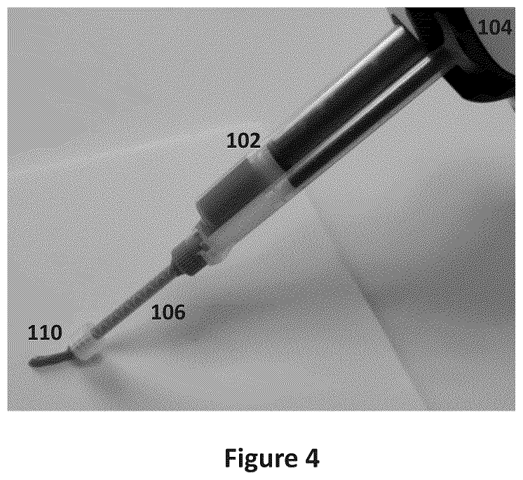

Referring now to FIG. 1, showing an image of an exemplary implementation device 100, according to some embodiments of the invention. In some embodiments, as mentioned above, the implementation device comprises a two-chambered cartridge 102 pre-filled with the two compounds of the bone cement. In some embodiments, the two-chambered cartridge is connected to an applicator 104, configured to push the two compounds out of the two chambered cartridge at the same time and into the mixing unit 106. FIG. 2 shows an image of an exemplary implementation device mounted and ready to be used, according to some embodiments of the invention. FIG. 3 shows an image of an exemplary implementation device being used and the two compounds being mixed inside the mixing unit 106 by being forced to pass through the sequence of mixing levels 108, according to some embodiments of the invention. FIG. 4 shows an image of an exemplary mixed cement 110 being applied from an exemplary implementation device, according to some embodiments of the invention.

### Exemplary Chemical Composition of the Bone Cement

In some embodiments, as mentioned above, the bone cement is an acrylic bone cement, whose ingredients have been divided in at least two components (A and B—see below) of viscose polymeric-based matter. In some embodiments, mixing the two parts starts a chemical cross-linking reaction, which turns the mixture of the two components into stiff matter. In some embodiments, as mentioned above, temperature and/or moisture accelerates the polymerization process.

Known acrylic cements are usually composed of polymerizing molecules: the liquid part, and non-covalently binding polymers—the solid powder part. In general, during mixing of the two parts, the liquid dissolves the powder and, after polymerization, the liquid monomers/dimers transform into the same polymers as the powder polymers and the rest of the components become a single mass consisting of the same polymer chains.

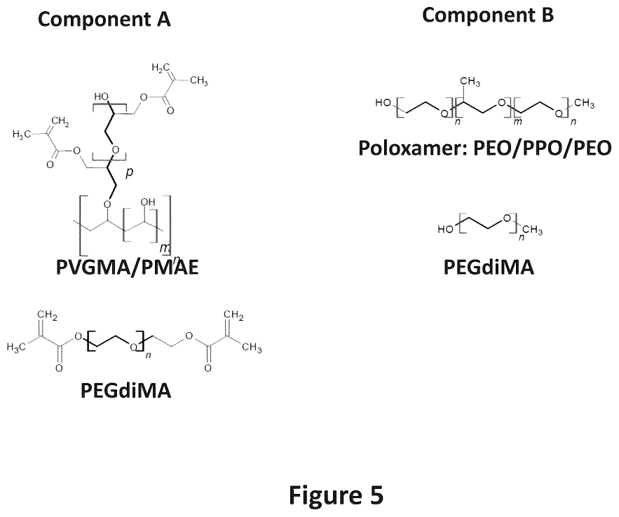

In some embodiments, in the bone cement of the present invention, the non-covalently binding part consists of an inert (in relation to the initiator) paste polymer. In some embodiments, the inert paste polymer does not have to undergo a dissolving process (or any other chemical process) in order to be ready. In some embodiments, a potential advantage of using an inert paste is that it can host the initiator molecules in a good and stable dispersion and, simultaneously, it can be easily and instantly mixed with the reactive paste part. In some embodiments, in order to allow efficient and stable mixing, the two (or more) components contain similar chemical, physical and mechanical properties. For example, the two components are based on poly-ether molecules in a variety of lengths, with or without different side groups. See FIG. 5, exemplary molecules of Component A and Component B, according to some embodiments of the invention.

### Component A

In some embodiments, Component A comprises molecules with active acrylic groups that can be cross-linked. In some embodiments, the viscosity and the hydrophobicity of the mixture are controlled by the type and ratio of its compounds (see table below).

In some embodiments, Component A is a mixture of one or more of the following exemplary methacrylic molecules:

Solid polymer, PVGMA: poly vinyl glycidyl methacrylate. A Polyvinyl alcohol (PVA)-based polymer modified with methacrylic groups, thereby generating a PVGMA. In some embodiments, the PVA can be at different degrees of hydrolyzation (from about 40% to about 99.9%), for example, 70%, 80% or 90%. In some embodiments, the methacrylic modification along the PVA can be at different ratios (for example from about 1:100 to about 1:1 or from 1:100 to 20:1) or as branches of poly glycidyl methacrylate ether (PGMAE) at different lengths and densities. In some embodiments, a pure PGMAE, a poly ether with methacrylate groups along the chain, is used instead of the PVGMA. In some embodiments, the PGMAE is synthetized by polymerization of GMA with its glycidyl group while leaving the methacrylate as a side group.

Liquid polymer, PEGdiMA: a solvent for the solid PVGMA that can be cross-linked. In some embodiments, it can be poly ethylene glycol di/mono-methacrylate, poly propylene glycol di/mono-methacrylate, poloxamer di/mono-methacrylate or another solvent. In some embodiments, the ratio between the liquid polymer and the solid polymer defines the viscosity of the mixture.

Paste short linker, bisGMA: a methacrylic molecule that can cross-link between all methacrylic groups of the mixture-solid, liquid and paste. In some embodiments, the molecule improves the cross-linking process and reinforces the final cross-linked matter. In some embodiments, it can be one or more of Bisphenol A glycidyl methacrylate (bisGMA), bisphenol A Ethylene Glycol Dimethacrylate (bisEMA), triethylene glycol dimethacrylate (TEGDMA), Urethane Dimethacrylate (UDMA) and others.

### Exemplary Additives to Component A

In some embodiments, in addition to the above methacrylic molecules, Component A can optionally comprise one or more of the following exemplary additives:

Radiopaque, BaSO₄: inert powder. In some embodiments, it enables viewing of the bone cement under X-ray. In some embodiments, it can be replaced with ZnO₂ or any other radiopaque material. In some embodiments, it can be added at 20% or at any amount between 10 and 50 percent of the final mass of the cement.

Inhibitor, HQ: hydroquinone. In some embodiments, it is used to stabilize the acrylic groups and potentially increases the shelf life of the product at room temperature. In some embodiments, it can be added at a concentration of 750 ppm or at any other concentration between 50 ppm and 2000 ppm. In some embodiments, exemplary inhibitors are: 4-methoxyphenol (MEHQ) and butylated hydroxytoluene (BHT).

Water: In some embodiments, it improves the cross-linking reaction. In some embodiments, water affects the rate and homogeneity of reaction. In some embodiments, water can be added at 10% of the total mass or at any other percentage between 0% (no water at all) and 40%.

### General Information Regarding Component A

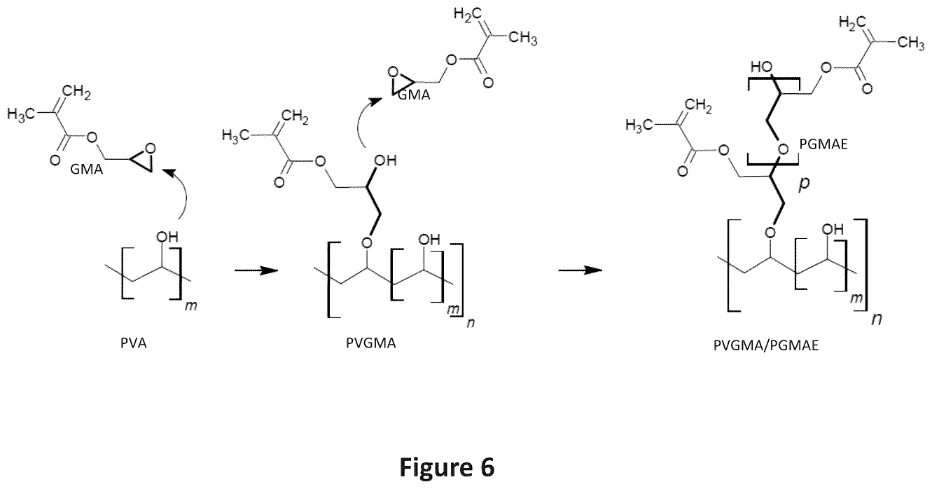

In some embodiments, the PGMAE is either part of the PVGMA or is an independent polymer in Component A. In some embodiments, a potential advantage of the PVGMA/PGMAE over the PVGMA alone is that the hydrophobicity of the PVGMA can be controlled by modifying the concentration of GMA along the PVA backbone chain. (See FIG. 6, an exemplary schematic chain reaction of addition of molecules, according to some embodiments of the invention.) In some embodiments, when modifying at a low concentration of GMA, the polymer contains long PVA regions, which increases the hydrophilicity of the molecule. In some embodiments, this potentially enables the absorption of water (hygroscopicity) while also allowing it to be soluble in water, which in some cases, is a feature not desired in implants but it is a feature of the bone cement of the present invention. In some embodiments, additionally, low concentrations of MA decrease the final strength of the cross-linked matter.

In some embodiments, in the PVGMA/PGMAE, branches of poly GMA-ether are grown as side chains along the PVGMA. In some embodiments, due to the high concentration of MA groups in the branches, the overall polymer is hydrophobic. In some embodiments, however, PVA regions remain along the PVGMA chains, and the ether groups along the PGMAE branches make the polymer hygroscopic. In some embodiments, the PGMAE branches are poly-ether therefore making the polymer also soluble in PEG (poly ethylene glycol) materials, which can contain water. In some embodiments, due to these characteristics, the PVGMA/PGMAE/PEG mixture can hold a certain amount of water. In some embodiments, the ability to absorb water allows the use of water soluble salts as initiators. In some embodiments, additionally, the high concentration of MA increases the final strength of the cross-linked matter.

In some embodiments, the viscosity of Component A before the mixing with Component B can be about 100 Pa·s, 300 Pa·s, or 700 Pa·s, for Low, Medium, or High viscosity cement, respectively.

### Component B

In some embodiments, Component B comprises the initiator compounds and optionally a paste polymeric matter. In some embodiments, the ratio between Component B and Component A is 1:10 or any other ratio between about 1:10 and about 1:1. In some embodiments, the ratio between the two components (A+B) affects the compression elasticity of the final cross-linked matter.

In some embodiments, Component B, which is a non-reactive polymer to the initiator, meets a number of requirements: in some embodiments it belongs to the same family of polymers as Component A (the polymerizing part), in order to create good van-der-Waals forces and polar bonds between the molecules of the two parts; in some embodiments it has a Tg (glass transition temperature) higher than 45 degrees Celsius in order to enable holding of the initiator's powder steadily (lower Tg is possible but will require refrigerated storage at temperature below the Tg); in some embodiments it is characterized by being a paste of matter, to ease mixing with Component A by mixing flow; in some embodiments it is slightly hygroscopic, in order to hold a small amount of water which, in some embodiments, is required for the initiation system.

In some embodiments, as long as these conditions are maintained, the amount and type of the non-reactive polymer (Component B) can be easily changed according to the desired final properties of the bone cement. For example, increasing the amount of the non-reactive polymer, or using more flexible molecules, will increase the elasticity of the cured matter, but also may decrease its final strength. In other embodiments, by using more hygroscopic polymers, it will absorb more water, which will lead to faster activation of the initiator and acceleration of the polymerization process and hardening of the matter.

In some embodiments, since the non-reactive polymer is not covalently bound to the polymers in Compound A in the final mixed bone cement, it can be replaced with a bio-degradable polymer. In this case, at the beginning after implantation, the non-reactive polymer acts as a load bearer, preventing mechanical collapse of the implant inside the body of the patient. Later on, the bio-degradable polymer starts decomposing, providing space for new bone to grow.

In some embodiments, the viscosity of Component B, before mixing with Component A, can be about 100 Pa·s, 300 Pa·s, or 700 Pa·s, for Low, Medium, or High viscosity cement, respectively.

In some embodiments, Component B comprises one or more of the following:

Polymeric filler, for example Poloxamer: In some embodiments, this paste matter contains no methacrylic groups and does not covalently bind to the methacrylic matrix, and therefore it can be placed with the initiators. In some embodiments, it has two roles: first as a medium for carrying and distributing the initiation molecules, and second to improve the compression elasticity of the final cross-linked matter. For example, the compression elasticity of bone is between 10 MPa and 500 MPa for osteoporotic and healthy bone, whereas the elasticity of common cement is about 1800 MPa. It has been shown that the stiffeners of the cement, compared to the bone, may cause fractions to the weak adjacent bones. In some embodiments, the bone cement of the present invention is characterized by an elasticity level similar to that of the bone, for example between about 250 MPa and about 500 MPa. In some embodiments, its viscosity is similar to that of Component A, and its structure allows good van-der-Waals forces and polar connections with Component A's polymers. In some embodiments, the polymeric filler can be, for example, one or more of: Pluronic P123, P105, P85, any other paste poloxamers, paste PEG1000, paste PPG8000, a mixture of short liquid and long solid PEGs PPGs, poloxamers, or other fillers.

In some embodiments, the polymeric filler can be a viscous acrylic polymer, for example: short poly butyl acrylate, poly butyl acrylate-co-hydroxyethyl methacrylate, poly butyl acrylate-co-acrylic acid, poly butyl acrylate-co-vinyl acetate, or others. In some embodiments, a specific filler is chosen according to the required hydrophobicity and/or viscosity of the material, for example 10-50 units of pure poly butyl acrylate for medium viscosity (up to 350 Pa·s at 25° C.) hydrophobic polymer, or 50-90 units for high viscosity (up to 1000 Pa·s at 25° C.) polymer. A copolymer of butyl-co-hydroxyl methacrylate composed of 10-20% 2-Hydroxyethyl methacrylate for semi-hydrophilic polymer, or a copolymer of butyl-co-acrylic acid composed of 40-70% acrylic acid for high hydrophilic polymer.

### Exemplary Use of Bio-Degradable Filler

In some embodiments, as mentioned above, the polymeric filler is only part of the implant matter, and not covalently bound to it. In some embodiments, using a bio-degradable polymer, such as polyglycolide (PGA), poly(L-lactide) (PLLA), or other polyesters as fillers, will lead over time to the formation of cavities within the implant, due to the bio-degradation of the material. In some embodiments, a potential advantage of using biodegradable fillers is that these cavities are a good substrate for bone tissue, and will therefore encourage bone regrowth into them. In some embodiments, the newly grown bone helps stabilize and integrate the implant and the bone.

Exemplary Use of cross-likable Filler

In some embodiments, the filler polymer comprises carbon-carbon Pi bonds that react with the acrylic radicals in the mixture, which are then cross-linked with part-A polymer. In some embodiments, the polymer can be poly propylene fumarate (PPF).

Initiation system: Since the bone cement mixture is configured to comprise slightly hydrophilic properties, hydrophilic initiation molecules are and can be used. In some embodiments, a potential advantage of having a bone cement configured as such is that the cross-linking-hardening reaction can be accelerated in a moist environment, for example, such as the one at the location of implantation inside the body.

### Exemplary Initiators

Initiator, NPS: In some embodiments, an exemplary initiator molecule is one or more of: sodium persulfate (NPS) or others like it, ammonium persulfate (APS) or potassium persulfate (KPS). In some embodiments, each initiator has a different solubility and decomposition constant, which affects the rate of initiation and cross-linking, therefore allowing the user to choose the adequate initiator according to his needs. In some embodiments, for example, using a molar ratio of 1:50 of NPS to MA groups, with 7% of water will lead to a curing time of 3 minutes at room temperature, while replacing the NPS with KPS will increase the curing time to about 15 minutes. In some embodiments, the concentration of the initiator is 1:50 moles in relation to the methacrylic groups, or any other ratio between about 1:10 and about 1:500.

In some embodiments, a less water-soluble initiator can be used, for example: benzoyl peroxide (BPO) initiator.

In some embodiments, the polymerization process is characterized by a dual-timing polymerization reaction. In some embodiments, at the first part of the reaction, the polymerization reaction is a quick reaction that is not temperature-dependent, while at the second part of the reaction, the polymerization reaction is a slow reaction that is temperature dependent.

Co-initiator, Ascorbic Acid: In some embodiments, the persulfate initiation decomposes spontaneously at high temperatures (above 70° C.) and only slightly at room temperature. In order to increase decomposition at body temperature, a co-initiator can be used. In some embodiments, ascorbic acid is used as a co-initiator. In some embodiments, a potential advantage found by the inventors of using ascorbic acid as a co-initiator is that ascorbic acid is a good co-initiator for persulfate-based initiators, while they can also be safely stored together. In some embodiments, for example, the co-initiator is used in a ratio of 1:1 mole in relation to the initiator or any other ratios between about 0.1:1 and about 2:1 (co-initiator: initiator). In some embodiments, the ascorbic acid also functions as a lone initiator if a long reaction is required. In some embodiments, alternatively and/or additionally, Iron (III) can be used as a co-initiator in the same way. In some embodiments, for more hydrophobic mixtures the ascorbic acid can be replaced with Ascorbyl Palmitate or Ascorbyl Stearate. In some embodiments, for the potassium persulfate initiator, a 18-crown ether is used as a co-initiator, but in this case they cannot be stored together and the 18-crown ether is added to Component A.

In some embodiments, when using the less water-soluble initiator, for example PBO, a suitable activator is used, for example: the common dimethyl-p-toluidine (DMPT).

Stabilizer, sorbitol: In some embodiments, optionally, stabilizers are added in order to improve the stability and shelf-life of the ascorbic acid. In some embodiments, an exemplary stabilizer is sorbitol. In some embodiments, stabilizers are used, for example, in a 1:1 mole ratio in relation to the ascorbic acid or in any ratio between about 0.1:1 and about 2:1 (stabilizer: ascorbic acid).

Radiopaque material: In some embodiments, a radiopacifier, for example BaSO₄, is added totally or partly to component B. In some embodiments, the mixing with the filler stabilizes the mixture and prevents sinking of the radiopacifier material. In some embodiments, transferring the radiopacifier to Component B decreases the ratio between Component A to Component B to value, for example, between 4:1 and 1:1.

### Exemplary Mechanical Characteristics

In some embodiments, the viscosity of the mixed paste, which is the bone cement during working time can be set to about 100 Pa·s, 500 Pa·s, and 1000 Pa·s for high, medium or low viscosity cements, respectively. In some embodiments, the curing time/hardening time, which is the time it takes for the mixed matter to reach its stable non-flowing stage, is temperature-dependent and can be set to a number between about 1 minute and about 10 minutes at body temperature and from about 5 minutes to about 40 minutes at room temperature. In some embodiments, the compression strength and elasticity at curing time are from about 50 MPa to about 70 MPa and from about 250 MPa to about 500 MPa, respectively. In some embodiments, after 24 hours, the bone cement reaches its full cured state (hardening state), with a final compression strength and elasticity of from about 80 MPa to about 200 MPa and, from about 200 MPa to about 2000 MPa, respectively.

Exemplary Use of More than Two Components

In some embodiments, the implementation device/delivery device comprises more than 2 cartridges/containers, for example, it comprises 3 or 4 or more cartridges/containers. For example, the total ingredients of the bone cement can be divided into three cartridges as follows: in the first cartridge Component A as disclosed above; in the second cartridge the initiator and the polymer material that is inert to the initiator; and in the third cartridge polymer material (optionally the same polymer material as in the second cartridge) with an additive that is required for a specific intervention, for example a pharmaceutical, cells, or other additive. In some embodiments, a potential advantage of this is that implementation devices/delivery devices can be prepared in advance with the first two cartridges ready to use, and a third cartridge that can be used only when necessary, therefore still facilitating the work of the medical personnel as previously mentioned while providing versatility to the bone cement system.

In some embodiments, additionally, any of the ingredients of Component A and/or B can be divided into two or more groups and into distinct cartridges, which are then mixed in the implementation device/delivery device.

### Exemplary Improvement of Known Bone Cement (PMMA Cement, for Example) Initiation Systems

In some embodiments, known bone cements can be improved by using the components of the present invention. In some embodiments, for example, the persulfate-ascorbic acid (and other mentioned co-initiators) can be adjusted to the MMA/PMMA cements, to replace the commonly-used BPO/DMPT. In some embodiments, one way to do so, is by using Ascorbyl Palmitate or Ascorbyl Stearate instead of ascorbic acid, and adding 18-Crown ether to the potassium persulfate, which potentially improves its solubility in organic mediums. In some embodiments, the reaction will start upon mixing of all compounds together. In some embodiments, another way of improving known bone cements, is by changing the hydrophobicity of the MMA/PMMA mixture. In some embodiments, for example, adding hydroxyethyl methacrylate (HEMA) monomers to the MMA solution and Poly(ethyl methacrylate) solid polymer to the powder, will make the mixture more hydrophilic, thus enabling it to absorb water for initiating the peroxide-ascorbic acid initiation couple.

### Exothermic Characteristics

In some embodiments, the bone cement is supplied at a high viscosity level, so that most of the exothermic chemical bonds have already been formed, i.e., the reaction begins only with polymers and without monomers, therefore, less new chemical bonds are formed. In some embodiments, during the polymerization process, fewer chemical bonds are formed and therefore the overall reaction is less exothermic and therefore the temperature rises by only a few degrees. In some embodiments, low exothermic reactions allow adding temperature sensitive additives like antibiotics. In some embodiments, a potential advantage of low exothermic reactions is that they do not damage the surrounding tissue during the polymerization process. In some embodiments, additionally, a potential advantage of providing high viscosity level bone cement is a potential reduction in leakage and potential improvement of the control of the material's flow.

### Exemplary Additives

In some embodiments, as mentioned above, the low exothermic reaction and the slightly hydrophilic nature of the bone cement allows the addition of a variety of additives. In some embodiments, for example, pharmaceuticals and/or antibiotics are added to the components, which will potentially improve the healing process, and reduce the risk of complications due to infections. While most antibiotics are unstable at high temperature and cannot stand the exothermic reaction of PMMA cements, they will easily stand the low exothermic reactions of the bone cement of the present invention. In some embodiments, additional materials such as stem cells and bone growth factors, which are sensitive to temperature and/or need an aqueous environment, and which are not effective in regular cements, can be incorporated into the bone cement without concern.

### Exemplary Curing Indicator

In some embodiments, as mentioned above, each compound comprises a color that allows the user to assess the mixing process. In some embodiments, once the mixing is complete the mixed material comprises a homogenous color. In some embodiments, additionally, the bone cement comprises an intrinsic color indicator, which changes during the hardening process, i.e., the mixed components of the bone cement change color during the polymerization process. Therefore, unique colors of the different components are used to assess the mixing process, and variation of the color of the mixed components is used to assess the polymerization process. In some embodiments, a potential advantage of this is that it allows the physician to have real-time information on the hardening stage of the bone cement. In some embodiments, for example, the bone cement starts as light-orange at its viscose flow stage, and turns light-yellow at the final hardening stage.

### Exemplary Formulations

In some embodiments, the bone cement of the present invention can have the following exemplary formulations (it should be understood that the following formulations are just examples provided to allow a person having skills in the art to understand the invention and are not intended to be limiting in any way), according to the desired viscosity and the desired curing velocity:

| | Component | HF | HS | MF | MS | LF | LS | | HPBC |
|---|---|---|---|---|---|---|---|---|---|
| Component | PVGMA (g) | 5 | 5 | 4 | 4 | 3 | 3 | | 4 |
| A | PEGdiMA | 2 | 2 | 4 | 4 | 5 | 5 | TEGDMA | 5 |
| | and/or | | | | | | | | |
| | TEGDMA | | | | | | | | |
| | (g) | | | | | | | | |
| | BisPhA- | 2 | 2 | 1 | 1 | 1 | 1 | | 1 |
| | GMA (g) | | | | | | | | |
| | HQ (ppm) | 750 | 750 | 750 | 750 | 750 | 750 | | 750 |
| | H2O (ml) | 1 | 0.5 | 1 | 0.5 | 1 | 0.5 | (or) DMPT | 0.02 |
| | BaSO4 (g) | 2.5 | 2.5 | 2.5 | 2.5 | 2.5 | 2.5 | | 2.5 |
| Component | Pluronic | 1 | 1 | 1 | 1 | 1 | 1 | pButA- | 1 |
| B | p123* | | | | | | | co-X | |
| | and/or | | | | | | | | |
| | pButA-co-X | | | | | | | | |
| | (g) | | | | | | | | |
| | NPS (g) | 0.2 | 0.2 | 0.19 | 0.19 | 0.18 | 0.18 | (or) BPO | 0.01 |
| | AsAc (g) | 0.15 | 0.15 | 0.14 | 0.14 | 0.13 | 0.13 | | — |
| | Sorbitol (g) | 0.03 | 0.03 | 0.03 | 0.03 | 0.03 | 0.03 | | — |

H = High viscosity (about 700 Pa · s);
M = Medium viscosity (about 300 Pa · s);
L = Low viscosity (about 100 Pa · s);
F = Fast curing (about 1 minute at body temperature, about 5 minutes at room temperature);
S = Slow curing (about 5 minutes at body temperature, about 15 minutes at room temperature);
*Pluronic p123-PEG8000/Pluronic p105-PEG8000/PEG1000-PEG8000; HPBC = Hydrophobic mixture; X = hydroxyethyl or acetate or carboxylic acid.

### Exemplary Methods

In some embodiments, an exemplary method of use is as follows:

Non-cross-linked or slightly cross-linked bone augmentation material comprising at least one initiator is mixed in a (optionally) disposable mixing unit. In some embodiments, cross-linking of the polymers in the bone augmentation material begins only at the disposable mixing unit. In some embodiments, the mixed materials are delivered into the chosen location. In some embodiments, the polymerization process of the mixed material is accelerated due to exposure to stimuli, in this case, for example, the natural body heat of the patient and/or exposure to moisture.

### General Exemplary Medical Applications

In some embodiments, the bone cement is used for a variety of medical applications, for example, to support fractured bones (e.g. Kyphoplasty and Vertebroplasty or as a bone filler), for the anchoring of artificial joints (e.g. Fenestrated screw cement augmentation or Cemented arthroplasty).

### General Exemplary Non-Medical Applications

In some embodiments, the materials used as medical grade implants are used in non-medical applications. In some embodiments, the materials are non-medically graded materials. The methods described hereinabove are also applicable here.

Some non-limiting examples of non-medical use are: void filling for construction or aviation (where a lightweight material is needed), gluing substitute anchoring (screw anchor), art, crafting, prototype fabrication, temperature isolation and fluid filtration.

As used herein with reference to quantity or value, the term “about” means “within +20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A medical grade bone cement having at least two mixable components, the bone cement comprising:
a. a first component comprising at least 20% by weight of a first polymer material in which at least 40% of monomeric units comprise a methacrylate group; and
b. a second component comprises at least one initiator selected to cause cross-linking of said first polymer material, wherein a balance of said second component comprises at least 50% by weight of a second polymer material that does not react with said initiator; said second component being a paste.

2. The medical grade bone cement according to claim 1, wherein said first component and said second component are characterized by having similar viscosities within a factor of 10.

3. The medical grade bone cement according to claim 1, wherein said first component is characterized by at least one selected from the group consisting of:
a. is less than 10% by weight of monomers;
b. is monomer-free;
c. comprises at least from about 20% to about 80% by weight of said first polymer material;
d. comprises a solvent selected from the group consisting of poly ethylene glycol di/mono-methacrylate, poly propylene glycol di/mono-methacrylate, poloxamer di/mono-methacrylate and any combination thereof;
e. comprises a PEG/PPG based solvent;
f. comprises a linker selected from the group consisting of Bisphenol A Glycidyl Dimethacrylate (bisGMA), bisphenol A Ethylene Glycol Dimethacrylate (bisEMA), triethylene glycol dimethacrylate (TEGDMA), Ethylene glycol dimethacrylate (EGDMA), Butandiol diacrylate (BDDA), Urethane Dimethacrylate (UDMA) and any combination thereof;
g. comprises at least one additive selected from the group consisting of a radiopaque material, an inhibitor, water and any combination thereof;
h. is characterized by the ability to absorb water;
i. is characterized by an inability to absorb water.

4-5. (canceled)

6. The medical grade bone cement according to claim 1, wherein said second component comprises one or more of:
a. at least from about 40% to about 100% by weight of said second polymer material;
b. at least one co-initiator;
c. at least one stabilizer;
d. at least one additive.

7. The medical grade bone cement according to claim 1, wherein said bone cement is provided as a ready to use bone cement in a dedicated implementation device.

8. The medical grade bone cement according to claim 1, wherein said first polymer material comprises one or more of:
a. a Polyvinyl alcohol (PVA) based polymer modified with methacrylic groups;
b. said PVA based polymer modified with methacrylic groups is PVGMA;
c. 100% PGMAE.

9. (canceled)

10. The medical grade bone cement according to claim 8, wherein a methacrylate modification along said PVA is characterized by one or more of:
a. a ratio of from about 1:100 to about 20:1;
b. addition of branches of poly glycidyl methacrylate ether (PGMAE).

11. (canceled)

12. The medical grade bone cement according to claim 1, wherein a minimum quantity of PGMAE in said first polymer material has a 1:10 ratio of PGMAE/PVA and said PGMAE comprises a length of 4 units.

13-18. (canceled)

19. The medical grade bone cement according to claim 3, wherein one or more of the following provide said ability to absorb water:
a. ether groups between methacrylic functional groups;
b. PVA.

20-21. (canceled)

22. The medical grade bone cement according to claim 1, wherein said second polymer material is selected from the group consisting of Pluronic P123, Pluronic P105, Pluronic P85, paste PEG1000, paste PPG8000, mixture of short liquid and long solid PEGs PPGs, poloxamers and any combination thereof.

23. The medical grade bone cement according to claim 1, wherein said second polymer material is at least one selected from the group consisting of:
a. a bio-degradable polymer;
b. a bio-degradable polymer that are one or more of polyesters and polysaccharides;
c. a bio-degradable polymer that is a cross-linkable polymer configured to be part of the cross-linking polymerization process;
d. a bio-degradable polymer that is a cross-linkable polymer configured to be part of the cross-linking polymerization process; and wherein said cross-linkable polymer is poly propylene fumarate (PPF).

24-26. (canceled)

27. The medical grade bone cement according to claim 1, wherein said at least one initiator is selected from the group consisting of sodium persulfate (NPS), ammonium persulfate (APS), potassium persulfate (KPS) and any combination thereof.

28. (canceled)

29. The medical grade bone cement according to claim 6, wherein said co-initiator is one or more of ascorbic acid and a modified molecule based on ascorbic acid.

30-32. (canceled)

33. The medical grade bone cement according to claim 6, wherein said at least one additive is one or more of a pharmaceutical, antibiotics, stem cells and bone growth factors.

34. The medical grade bone cement according to claim 1, wherein a concentration of said initiator is from about 1:10 to about 1:500 in relation to methacrylic functional groups.

35. The medical grade bone cement according to claim 1, wherein a ratio between said first component and said second component is from about 1:10 to about 1:1.

36. The medical grade bone cement according to claim 1, wherein said medical grade bone cement, after being mixed, is characterized by at least one of the following:
a. having a low viscosity level of from about 10 Pa·s to about 100 Pa·s;
b. having a medium viscosity level of from about 100 Pa·s to about 500 Pa·s;
c. having a high viscosity level of from about 500 Pa·s to about 1500 Pa·s.

37-38. (canceled)

39. The medical grade bone cement according to claim 1, wherein said medical grade bone cement is characterized by at least one selected from the group consisting of:
a. a working time from about 1 min to about 40 min at room temperature;
b. a hardening time starting from about an end of said working time to about 24 hours;
c. achieving a non-deformable stage after from about 1 minute to about 5 minutes from about an end of said working time.

40-41. (canceled)

42. The medical grade bone cement according to claim 1, wherein a polymerization process is affected by one or more of temperature and moisture.

43. (canceled)

44. The medical grade bone cement according to claim 1, wherein at least one of the following is true:
a. exposure of said bone cement to temperatures of from about 35° C. to about 40° C. accelerates the polymerization process time by a factor of about 2 to about 5 compared to process time at temperatures of from about 20° C. to about 25° C.;
b. exposure of said bone cement to moisture accelerates the polymerization process time by a factor of about 2 to about 5;
c. concomitant exposure of said bone cement to moisture and to temperatures of from about 35° C. to about 40° C. accelerates the polymerization process time by a factor of about 2 to about 25.

45-46. (canceled)

47. The medical grade bone cement according to claim 1, wherein said medical grade bone cement comprises a color indicator for a polymerization process.

48. A method of polymerizing a bone cement, comprising providing said bone cement with two distinct polymerization reactions, wherein a first polymerization reaction is characterized by being a fast polymerization reaction that is not temperature dependent, while a second polymerization reaction is characterized by being a slow polymerization reaction that is temperature dependent.

49. A delivery device for a bone cement comprising:
a. a mixing unit comprising:
i. at least one cartridge comprising at least two chambers; each chamber from said at least two chambers containing a part of said bone cement; said at least one cartridge comprising a pushing mechanism of said parts of said bone cement; wherein said pushing mechanism is configured to move a same distance within said at least two chambers; and
ii. a mixer connected to said at least two chambers and configured to receive parts of said bone cement contained in said at least two chambers;
b. a control unit comprising at least one actuator for actuating at least one actuating mechanism to cause said parts of said bone cement contained in said at least two chambers to exit from said at least two chambers into said mixer;
c. a connector, connected on one side to said mixing unit and on a second side to said control unit; said connector configured to transmit actuation actions from said control unit to said mixing unit.

50. The delivery device according to claim 49, wherein said at least one actuating mechanism comprises one or more of hydraulic, mechanical and electrical actuation mechanisms.

51. The delivery device according to claim 49, wherein said pushing mechanism is a plunger configured to push said parts of said bone cement out of said at least two chambers.

52. The medical grade bone cement according to claim 1, wherein said balance of said second component comprises at least 10% by weight of said second polymer material that does not react with said initiator.
